(19) United States
(12) Patent Application Publication
(10) Pub. No.: US 2017/0035894 A1
(43) Pub. Date: Feb. 9, 2017

(54) DRUG DELIVERY CONJUGATES FOR TREATING RESISTANT CANCER AND FOR USE IN COMBINATION THERAPY

(71) Applicant: ENDOCYTE, INC., West Lafayette, IN (US)

(72) Inventors: Iontcho Radoslavov VLAHOV, West Lafayette, IN (US); Christopher Paul LEAMON, West Lafayette, IN (US); Joseph Anand REDDY, West Lafayette, IN (US)

(21) Appl. No.: 15/303,953

(22) PCT Filed: Apr. 14, 2015
(86) PCT No.: PCT/US15/25790

§ 371 Date: Oct. 13, 2016

Related U.S. Application Data
(60) Provisional application No. 61/979,344, filed on Apr. 14, 2014.
(60) Provisional application No. 62/057,473, filed on Sep. 30, 2014.

(51) Int. Cl. A61K 47/48; A61K 31/4745; A61K 39/395; A61K 31/357; A61K 31/704; A61K 31/282; A61K 33/24; A61K 31/337; C07K 16/22

(52) U.S. Cl. CPC A61K 47/48107; A61K 47/48246; A61K 31/337; A61K 31/4745; A61K 39/3955; C07K 16/22; A61K 31/704; A61K 31/282; A61K 33/24; A61K 31/357; C07K 2317/24; A61K 2039/505

(57) ABSTRACT

Described herein are drug delivery conjugates for targeted therapy. In particular, described herein are drug delivery conjugates that include polyvalent linkers comprising one or more unnatural amino acids that are useful for treating cancers and inflammatory diseases. In some embodiments, the cancer is selected from the group consisting of a carcinoma, a sarcoma, a lymphoma, Hodgekin's disease, a melanoma, a mesothelioma, Burkitts lymphoma, a nasopharyngeal carcinoma, a leukemia, and a myeloma.

### CROSS-REFERENCE TO RELATED APPLICATIONS

This application claims priority under 35 U.S.C. §119(e) to U.S. Provisional Application Ser. No. 61/979,344, filed Apr. 14, 2014 and U.S. Provisional Application Ser. No. 62/057,473, filed Sep. 30, 2014, the disclosures of both of which are expressly incorporated by reference herein.

### TECHNICAL FIELD

The invention described herein pertains to drug delivery conjugates for treating resistant cancer. In addition, the invention described herein pertains to drug delivery conjugates that enhance the efficacy of other anti-cancer agents.

### BACKGROUND

The mammalian immune system provides a means for the recognition and elimination of pathogenic cells, such as tumor cells, and other invading foreign pathogens. While the immune system normally provides a strong line of defense, there are many instances where pathogenic cells, such as cancer cells, and other infectious agents evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they lack sufficient selectivity to preferentially destroy pathogenic cells, and therefore, may also harm normal host cells, such as cells of the hematopoietic system, and other non-pathogenic cells. The adverse side effects of these anticancer drugs highlight the need for the development of new therapies selective for pathogenic cell populations and with reduced host toxicity.

It has been discovered herein that drug delivery conjugates that include polyvalent linkers formed from one or more unnatural amino acids are efficacious in treating pathogenic cell populations, and exhibit low host animal toxicity.

### SUMMARY

In one illustrative embodiment, the disclosure provides a method for treating cancer in a host animal, the method comprising the step of administering to the host animal a therapeutically effective amount of a compound of the formula

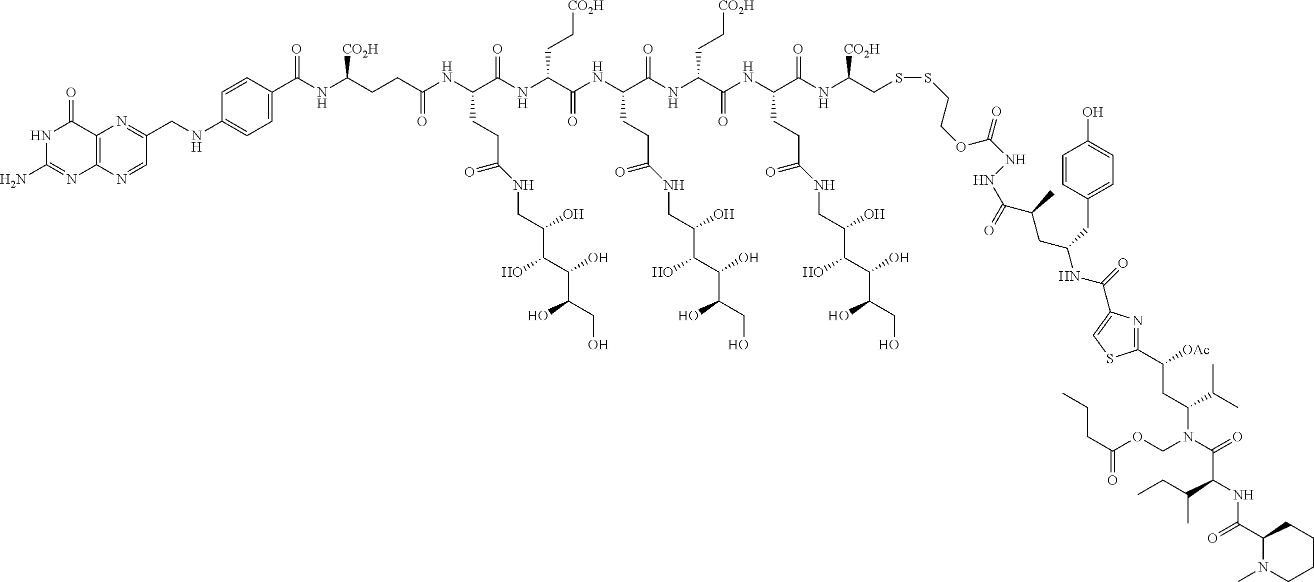 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof,

in combination with a therapeutically effective amount of at least one additional anti-cancer agent.

In another illustrative embodiment, the disclosure provides a use of a compound of the formula

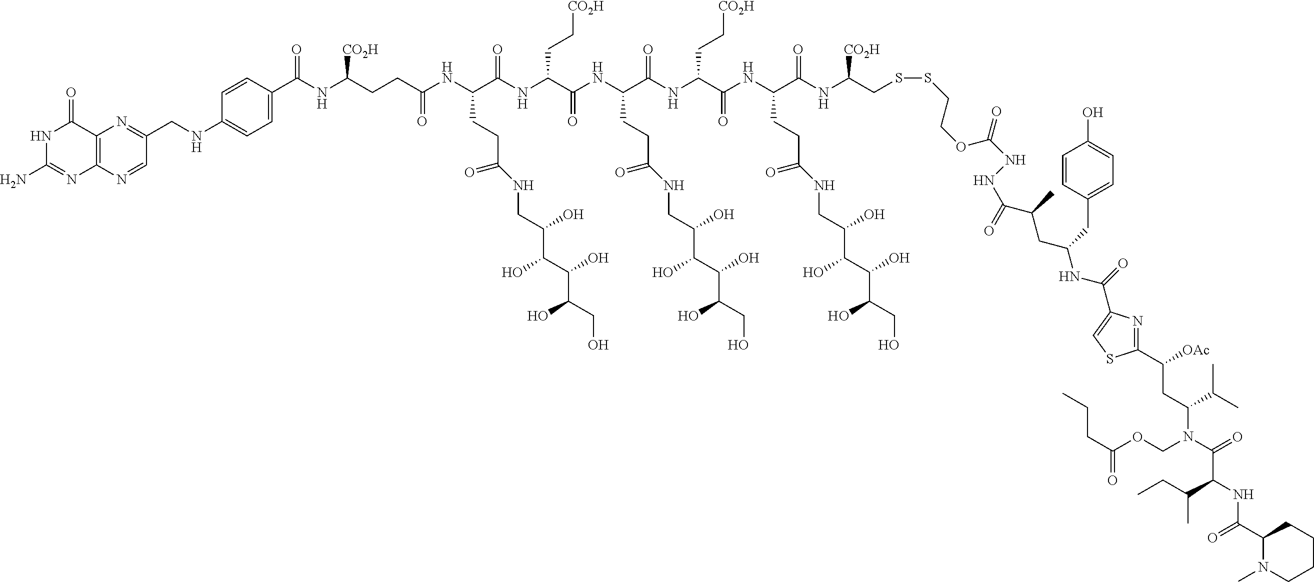 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof, in combination with a therapeutically effective amount of at least one additional anti-cancer agent, for treating a cancer in a patient.

In some embodiments, the cancer is selected from the group consisting of a carcinoma, a sarcoma, a lymphoma, Hodgekin's disease, a melanoma, a mesothelioma, Burkitt's lymphoma, a nasopharyngeal carcinoma, a leukemia, and a myeloma. In some embodiments, the cancer is selected from the group consisting of oral cancer, thyroid cancer, endometrial cancer, endocrine cancer, skin cancer, gastric cancer, esophageal cancer, laryngeal cancer, pancreatic cancer, colon cancer, bladder cancer, bone cancer, ovarian cancer, cervical cancer, uterine cancer, breast cancer, testicular cancer, prostate cancer, rectal cancer, kidney cancer, endometrial cancer, liver cancer, and lung cancer. In some embodiments, the cancer is ovarian cancer. In some embodiments, the cancer is non-small cell lung cancer. In some embodiments, the cancer is endometrial cancer. In some embodiments, the cancer is triple negative breast cancer. In some embodiments, the cancer is breast cancer. In some embodiments, the cancer is lung cancer.

In some embodiments, the additional anti-cancer agent has a mode of action selected from the group consisting of intercalating or inhibiting macromolecular biosynthesis, inhibiting progression of the enzyme topoisomerase II, relaxing DNA supercoils, inhibiting transcription, stabilizating topoisomerase II complexes, preventing DNA double helices from being resealed, inhibiting DNAreplication, inducing histone eviction from chromatin; crosslinking DNA, eliciting DNA repair mechanisms, which in turn activate apoptosis; inhibiting angiogenesis; inhibiting topoisomerase-1; binding and/or stabilizing microtubules, preventing physiological microtubule depolymerisation/disassembly leading to apoptosis, phosphorylating oncoprotein bcl-2 leading to apoptosis unblocking, suppressing microtubule dynamic assembly and disassembly; inhibiting spindle function, suppressing microtubule dynamics, suppressing microtubule detachment from centrosomes; and interfering with DNA repair, and combinations thereof. In some embodiments, the additional anti-cancer agent is selected from the group consisting of doxorubicin (DOXIL), cisplatin, bevacizumab (Avastin), topotecan, eribulin mesylate, docetaxel, paclitaxel, and carboplatin, and pharmaceutically acceptable salts of the foregoing, and combinations thereof. In some embodiments, the additional anti-cancer agent is doxorubicin (DOXIL), or a pharmaceutically acceptable salt thereof. In some embodiments, the additional anti-cancer agent is cisplatin, or pharmaceutically acceptable salt thereof. In some embodiments, the additional anti-cancer agent is bevacizumab (Avastin), or a pharmaceutically acceptable salt thereof. In some embodiments, the additional anti-cancer agent is topotecan, or a pharmaceutically acceptable salt thereof. In some embodiments, the additional anti-cancer agent is docetaxel, or a pharmaceutically acceptable salt thereof. In some embodiments, the additional anti-cancer agent is paclitaxel, or a pharmaceutically acceptable salt thereof. In some embodiments, the additional anti-cancer agent is carboplatin, or a pharmaceutically acceptable salt thereof. In some embodiments, the additional anti-cancer agent is eribulin mesylate.

In another illustrative and non-limiting embodiment of the invention, described herein are compounds of the formula

B-L-Dₓ

wherein each of B, L, D, and x are as defined in the various embodiments and aspects described herein.

In another embodiment, pharmaceutical compositions containing one or more of the compounds are also described herein. In one aspect, the compositions include a therapeutically effective amount of the one or more compounds for treating a patient with cancer, inflammation, and the like. It is to be understood that the compositions may include other components and/or ingredients, including, but not limited to, other therapeutically active compounds, and/or one or more carriers, diluents, excipients, and the like, and combinations thereof. In another embodiment, methods for using the compounds and pharmaceutical compositions for treating patients or host animals with cancer, inflammation, and the like are also described herein. In one aspect, the methods include the step of administering one or more of the compounds and/or compositions described herein to a patient with cancer, inflammation, and the like. In another aspect, the methods include administering a therapeutically effective amount of the one or more compounds and/or compositions described herein for treating patients with cancer, inflammation, and the like. In another embodiment, uses of the compounds and compositions in the manufacture of a medicament for treating patients with cancer, inflammation, and the like are also described herein. In one aspect, the medicaments include a therapeutically effective amount of the one or more compounds and/or compositions for treating a patient with cancer, inflammation, and the like.

### BRIEF DESCRIPTION OF THE DRAWINGS

FIG. 1A shows in vivo activity of EC1456 against KB tumors in nu/nu mice dosed at 1 μmol/kg three times per week (M/W/F) TIW) for two consecutive weeks (), compared to EC1456 co-dosed with EC0923 at 100 μmol/kg (▴), and untreated (PBS) controls (▪). The dotted vertical line represents the day of the final dose. FIG. 1B shows that EC1456 did not result in any observable whole animal toxicity as determined by animal body weight.

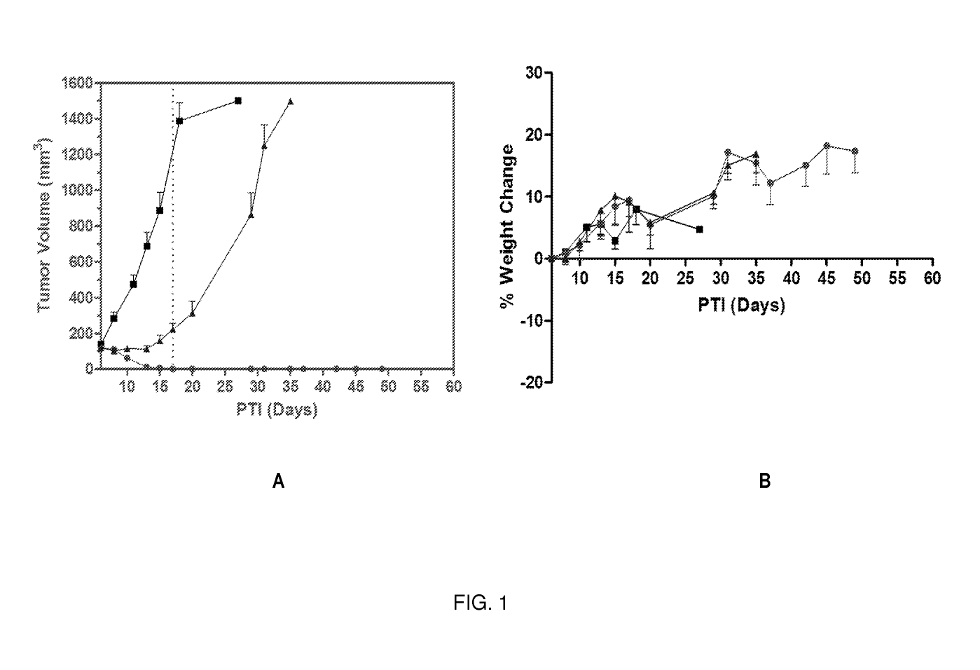

FIG. 2A shows the activity of EC1456 against established subcutaneous MDA-MB-231 tumors. Animals bearing s.c. MDA-MB-231 tumors (94-145 mm³) were treated i.v. starting on Day 17 with 2 μmol/kg (panel A) of EC1456 (), three times per week (M/W/F) for a 2 week period, and compared to untreated animals (▪), as shown in FIG. 5A. N=5 animals per cohort. Dotted vertical line=day of final dose. FIG. 2B shows that EC1456 did not cause gross whole animal toxicity as determined by % weight change.

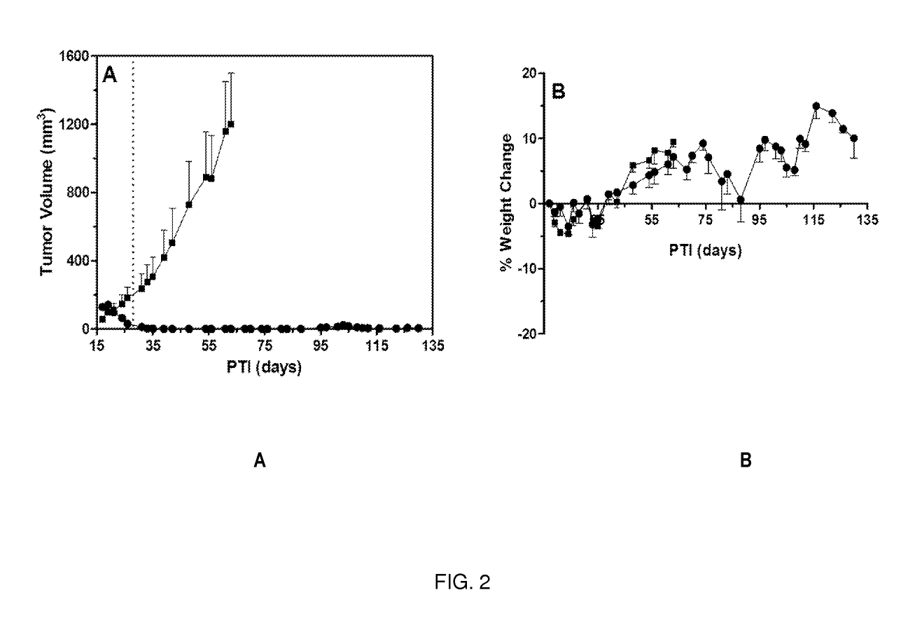

FIG. 3A shows the activity of EC1456 in animals bearing s.c. KB-CR2000 (cisplatin resistant) tumors (98-148 mm3), where EC1456 was administered i.v. starting on Day 6 with 2 μmol/kg (), three times per week (M/W/F) for a 2 week period, or with 3 mg/kg of cisplatin (▴), twice per week (T/Th) for a 2 week period, and compared to untreated controls (▪), N=5 animals per cohort. Dotted vertical line=day of final dosing day. FIG. 3B shows that EC1456 did not exhibit significant host animal toxicity. In contrast, cisplatin treatment resulted in substantial host animal toxicity during the dosing period.

FIG. 4 shows the maximum tolerated dose (MTD) of EC1456 compared to vehicle controls. Vehicle control (▪), EC1456 at 0.33 μmol/kg (), EC1456 at 0.41 μmol/kg (▴), EC1456 at 0.51 μmol/kg (▾), and EC1456 at 0.67 μmol/kg (♦).

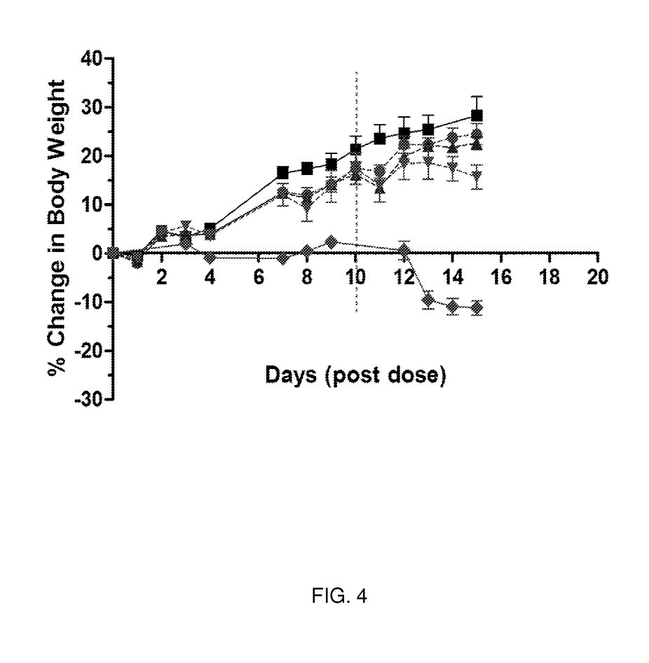

FIG. 5A shows the activity of EC1456 with DOXIL in animals bearing s.c. vinca resistance KB-DR150 tumors, where the dotted vertical line=day of final dosing day; ▪ is the Control; ▾ is EC1456 administered 2 μmol/kg, TIW×2; is DOXIL administered at 5 mg/kg, BIW×2; ◯ is EC1456 administered at 2 μmol/kg, TIW×2+DOXIL 5 mg/kg, BIW×2. FIG. 5B shows % weight change over time for the same experiment.

FIG. 6A shows the activity of EC1456 with cisplatin in animals bearing s.c. M109 tumors, where the dotted vertical line=day of final dosing day; ▪ is the Control; ▾ is EC1456 administered 2 μmol/kg, TIW×2; □ is cisplatin administered at 3 mg/kg, BIW×2; Δ is EC1456+cisplatin. FIG. 6B shows % weight change over time for the same experiment.

FIG. 7A shows the activity of EC1456 with cisplatin in animals bearing s.c. KB tumors (oral epidermoid carcinoma), where the dotted vertical line=day of final dosing day; is the Control; ▾ is EC1456 administered 1 μmol/kg, BIW×2; ▪ is EC1456 administered at 1 μmol/kg, BIW×2+cisplatin administered at 3 mg/kg, BIW×3; ▴ is cisplatin administered at 3 mg/kg, BIW×3. FIG. 7B shows % weight change over time for the same experiment.

FIG. 8A shows the activity of EC1456 with bevacizumab in animals bearing s.c. KB tumors, where the dotted vertical line=day of final dosing day; (a) is the Control; (b) is EC1456 administered 1 μmol/kg, TIW×2; (c) is EC1456 administered at 1 μmol/kg, TIW×2+avastin administered at 5 mg/kg, BIW×2; (d) is avastin administered at 5 mg/kg, BIW×2. FIG. 8B shows % weight change over time for the same experiment.

FIG. 9A shows the activity of EC1456 with topotecan in animals bearing s.c. P-1606 KB tumors, where the dotted vertical line=day of final dosing day; (a) is the Control; (b) is EC1456 administered 1 μmol/kg, BIW×2; (c) is EC1456 administered at 1 μmol/kg, BIW×2+topotecan administered at 5 mg/kg, BIW×2; (d) is topotecan administered at 5 mg/kg, BIW×2. FIG. 9B shows % weight change over time for the same experiment.

FIG. 10A shows the activity of EC1456 with topotecan in animals bearing s.c. P-1609 KB tumors, where the dotted vertical line=day of final dosing day; (a) is the Control; (b) is EC1456 administered 1 μmol/kg, TIW×5; (c) is topotecan administered at 5 mg/kg, TIW×5; (d) is EC1456 administered at 1 μmol/kg, TIW×5+topotecan administered at 5 mg/kg, TIW×5. FIG. 10B shows % weight change over time for the same experiment.

FIG. 11A shows the activity of EC1456 with docetaxel in animals bearing s.c. P-1609 KB tumors, where the dotted vertical line=day of final dosing day; (a) is the Control; (b) is EC1456 administered 1 μmol/kg, TIW×5; (c) is docetaxel administered at 7 mg/kg, BIW×3; (d) is EC1456 administered at 1 μmol/kg, TIW×5+docetaxel administered at 7 mg/kg, BIW×3. FIG. 11B shows % weight change over time for the same experiment.

FIG. 12A shows the activity of EC1456 with carboplatin in animals bearing s.c. P-1635 KB tumors, where the dotted vertical line=day of final dosing day; is the Control; ▴ is EC1456 administered 1 μmol/kg, TIW×2; ◯ is carboplatin administered at 50 mg/kg TIW; □ is EC1456 administered 1 μmol/kg, TIW+carboplatin administered at 50 mg/kg TIW. FIG. 12B shows % weight change over time for the same experiment.

FIG. 13 shows the activity of EC1456 with carboplatin and paclitaxel in animals bearing s.c. KB tumors, where the dotted vertical line=day of final dosing day; ▪ is the Control; ▴ is EC1456 administered 1 μmol/kg, BIW×2; ▾ is carboplatin administered at 30 mg/kg, BIW×2+paclitaxel, 10 mg/kg, BIW×2; is EC1456 administered at 1 μmol/kg, BIW×2+carboplatin 30 mg/kg, BIW×2+paclitaxel, 10 mg/kg, BIW×2.

FIG. 14 shows the activity of EC1456 with paclitaxel in animals bearing s.c. ST040 tumors, where the dotted vertical line=day of final dosing day; ▪ is the Control; ▾ is paclitaxel administered at 15 mg/kg, SIW×2; (a) is EC1456 administered at 1.5 μmol/kg BIW×2+paclitaxel administered at 15 mg/kg, SIW×2; (b) is EC1456 administered at 3 μmol/kg BIW×2+paclitaxel administered at 15 mg/kg, SIW×2.

FIG. 15 shows the activity of EC1456 with eribulin mesylate in animals bearing s.c. ST502 tumors, where the dotted vertical line=day of final dosing day; ▪ is the Control; ▾ is eribulin mesylate administered 1 mg/kg, SIW×2; (a) is EC1456 administered at 2 μmol/kg BIW×2+eribulin mesylate administered 1 mg/kg; (b) is EC1456 administered at 4 μmol/kg BIW×2+eribulin mesylate administered 1 mg/kg.

FIG. 16 shows the activity of EC1456 with eribulin mesylate in animals bearing s.c. ST738 tumors, where the dotted vertical line=day of final dosing day; ▪ is the Control; ▾ is eribulin mesylate administered 1 mg/kg, SIW×2; (a) is EC1456 administered at 2 μmol/kg BIW×2+eribulin mesylate administered 1 mg/kg; (b) is EC1456 administered at 4 μmol/kg BIW×2+eribulin mesylate administered 1 mg/kg.

FIG. 17 shows the activity of EC1456 with paclitaxel in animals bearing s.c. ST024 tumors, where the dotted vertical line=day of final dosing day; ▪ is the Control; ▾ is paclitaxel administered at 15 mg/kg, SIW×2; (a) is EC1456 administered at 2 μmol/kg BIW×2+paclitaxel administered at 15 mg/kg, SIW×2; (b) is EC1456 administered at 4 μmol/kg SIW×2+paclitaxel administered at 15 mg/kg, SIW×2.

FIG. 18 shows the activity of EC1456 with paclitaxel in animals bearing s.c. LU1147 NSCLC tumors, where the dotted vertical line=day of final dosing day; ▪ is the Control; ▾ is docetaxel administered at 15 mg/kg, SIW; (a) is EC1456 administered at 2 μmol/kg BIW×2+docetaxel administered at 15 mg/kg, SIW×2; (b) is EC1456 administered at 4 μmol/kg SIW×2+docetaxel administered at 15 mg/kg.

FIG. 19 shows the activity of EC1456 with paclitaxel in animals bearing s.c. LU2505 NSCLC tumors, where the dotted vertical line=day of final dosing day; ▪ is the Control; ▾ is docetaxel administered at 15 mg/kg, SIW; (a) is EC1456 administered at 2 μmol/kg BIW×2+docetaxel administered at 15 mg/kg, SIW×2; (b) is EC1456 administered at 4 μmol/kg SIW×2+docetaxel administered at 15 mg/kg.

### DETAILED DESCRIPTION

Several illustrative embodiments of the invention are described by the following enumerated clauses:

1. A method for treating cancer in a host animal, the method comprising the step of administering to the host animal a therapeutically effective amount of a compound of the formula

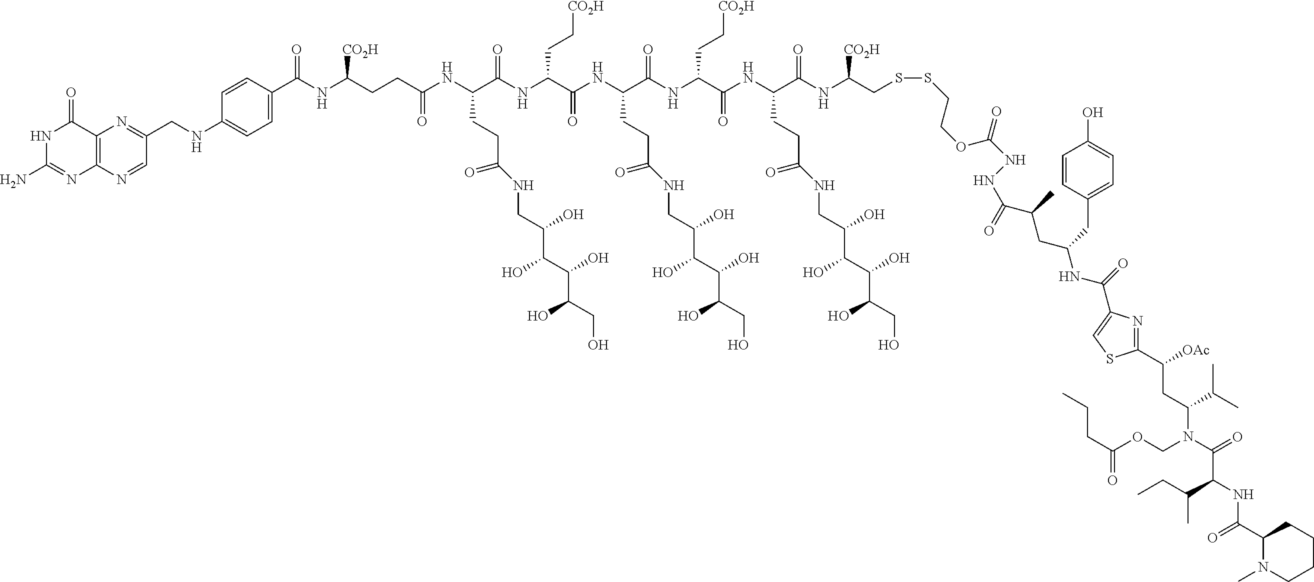 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof,

in combination with a therapeutically effective amount of at least one additional anti-cancer agent.

2. The method of clause 1, wherein the cancer is selected from the group consisting of a carcinoma, a sarcoma, a lymphoma, Hodgekin's disease, a melanoma, a mesothelioma, Burkitt's lymphoma, a nasopharyngeal carcinoma, a leukemia, and a myeloma.

3. The method of clause 1 or 2, wherein the cancer is selected from the group consisting of oral cancer, thyroid cancer, endometrial cancer, endocrine cancer, skin cancer, gastric cancer, esophageal cancer, laryngeal cancer, pancreatic cancer, colon cancer, bladder cancer, bone cancer, ovarian cancer, cervical cancer, uterine cancer, breast cancer, testicular cancer, prostate cancer, rectal cancer, kidney cancer, endometrial cancer, liver cancer, and lung cancer.

4. The method of any one of clauses 1 to 3, wherein the cancer is ovarian cancer.

5. The method of any one of clauses 1 to 3, wherein the cancer is non-small cell lung cancer.

6. The method of any one of clauses 1 to 3, wherein the cancer is endometrial cancer.

7. The method of any one of clauses 1 to 3, wherein the cancer is triple negative breast cancer.

8. The method of any one of clauses 1 to 3, wherein the cancer is breast cancer.

9. The method of any one of clauses 1 to 3, wherein the cancer is lung cancer.

10. The method of any one of clauses 1 to 9, wherein the additional anti-cancer agent is selected from the group consisting of doxorubicin (DOXIL), cisplatin, bevacizumab (Avastin), topotecan, eribulin mesylate, docetaxel, paclitaxel, and carboplatin, or a pharmaceutically acceptable salt thereof.

11. The method of any one of clauses 1 to 10, wherein the additional anti-cancer agent is selected from the group consisting of eribulin mesylate, docetaxel and paclitaxel, or a pharmaceutically acceptable salt thereof.

12. The method of any one of clauses 1 to 9, wherein the additional anti-cancer agent is doxorubicin (DOXIL), or a pharmaceutically acceptable salt thereof.

13. The method of any one of clauses 1 to 9, wherein the additional anti-cancer agent is cisplatin, or pharmaceutically acceptable salt thereof.

14. The method of any one of clauses 1 to 9, wherein the additional anti-cancer agent is bevacizumab (Avastin), or a pharmaceutically acceptable salt thereof.

14. The method of any one of clauses 1 to 9, wherein the additional anti-cancer agent is eribulin mesylate.

16. The method of any one of clauses 1 to 9, wherein the additional anti-cancer agent is docetaxel, or a pharmaceutically acceptable salt thereof.

17. The method of any one of clauses 1 to 9, wherein the additional anti-cancer agent is paclitaxel, or a pharmaceutically acceptable salt thereof.

18. The method of any one of clauses 1 to 9, wherein the additional anti-cancer agent is carboplatin, or a pharmaceutically acceptable salt thereof.

19. Use of a compound of the formula

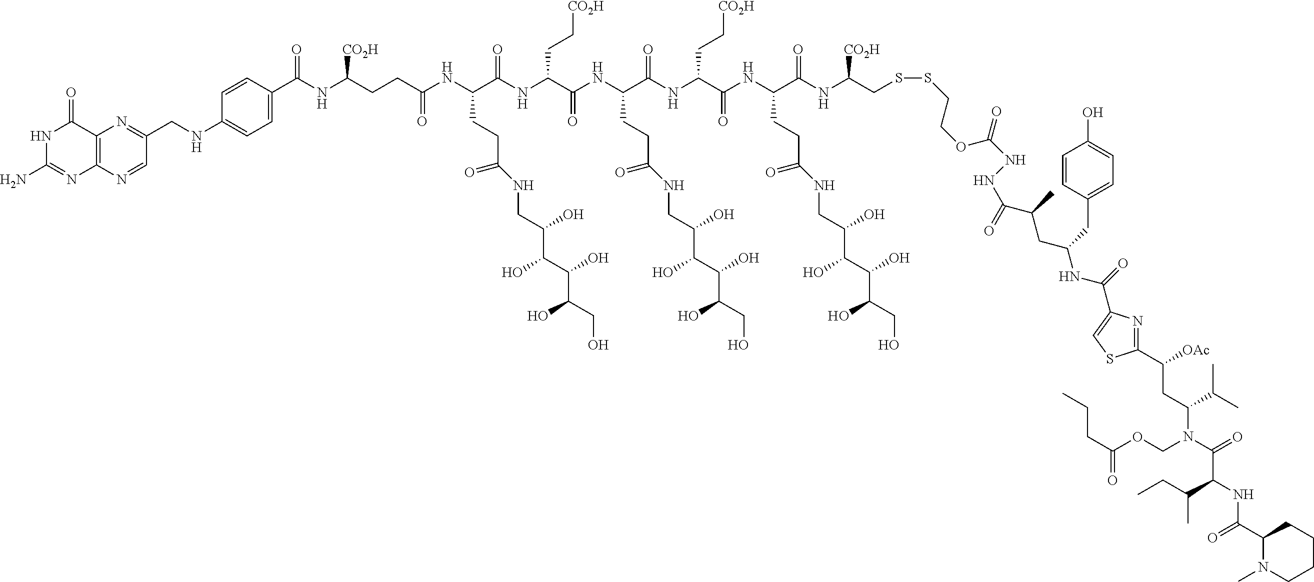 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof, in combination with a therapeutically effective amount of at least one additional anti-cancer agent, for treating a cancer in a patient.

20. The use of clause 19, wherein the cancer is selected from the group consisting of a carcinoma, a sarcoma, a lymphoma, Hodgekin's disease, a melanoma, a mesothelioma, Burkitt's lymphoma, a nasopharyngeal carcinoma, a leukemia, and a myeloma.

21. The use of clause 19 or 20, wherein the cancer is selected from the group consisting of oral cancer, thyroid cancer, endometrial cancer, endocrine cancer, skin cancer, gastric cancer, esophageal cancer, laryngeal cancer, pancreatic cancer, colon cancer, bladder cancer, bone cancer, ovarian cancer, cervical cancer, uterine cancer, breast cancer, testicular cancer, prostate cancer, rectal cancer, kidney cancer, endometrial cancer, liver cancer, and lung cancer.

22. The use of any one of clauses 19 to 21, wherein the cancer is ovarian cancer.

23. The use of any one of clauses 19 to 21, wherein the cancer is non-small cell lung cancer.

24. The use of any one of claims 19 to 21, wherein the cancer is endometrial cancer.

25. The use of any one of clauses 19 to 21, wherein the cancer is triple negative breast cancer.

26. The use of any one of clauses 19 to 21, wherein the cancer is breast cancer.

27. The use of any one of clauses 19 to 21, wherein the cancer is lung cancer.

28. The method of any one of clauses 19 to 27, wherein the additional anti-cancer agent is selected from the group consisting of doxorubicin (DOXIL), cisplatin, bevacizumab (Avastin), topotecan, eribulin mesylate, docetaxel, paclitaxel, and carboplatin, or a pharmaceutically acceptable salt thereof.

29. The method of any one of clauses 19 to 28, wherein the additional anti-cancer agent is selected from the group consisting of eribulin mesylate, docetaxel and paclitaxel, or a pharmaceutically acceptable salt thereof.

30. The use of any one of clauses 19 to 28, wherein the additional anti-cancer agents is a combination of paclitaxel and carboplatin, or a pharmaceutically acceptable salt thereof.

31. The use of any one of clauses 19 to 28, wherein the additional anti-cancer agent is doxorubicin (DOXIL), or a pharmaceutically acceptable salt thereof.

32. The use of any one of clauses 19 to 28, wherein the additional anti-cancer agent is cisplatin, or pharmaceutically acceptable salt thereof.

33. The use of any one of clauses 19 to 28, wherein the additional anti-cancer agent is bevacizumab (Avastin), or a pharmaceutically acceptable salt thereof.

34. The use of any one of clauses 19 to 28, wherein the additional anti-cancer agent is topotecan, or a pharmaceutically acceptable salt thereof.

35. The use of any one of clauses 19 to 28, wherein the additional anti-cancer agent is docetaxel, or a pharmaceutically acceptable salt thereof.

36. The use of any one of clauses 19 to 28, wherein the additional anti-cancer agent is paclitaxel, or a pharmaceutically acceptable salt thereof.

37. The use of any one of clauses 19 to 28, wherein the additional anti-cancer agent is carboplatin, or a pharmaceutically acceptable salt thereof.

38. The use of any one of clauses 19 to 28, wherein the additional anti-cancer agent is eribulin mesylate.

Several illustrative embodiments of the invention are described by the following clauses:

A compound of the formula

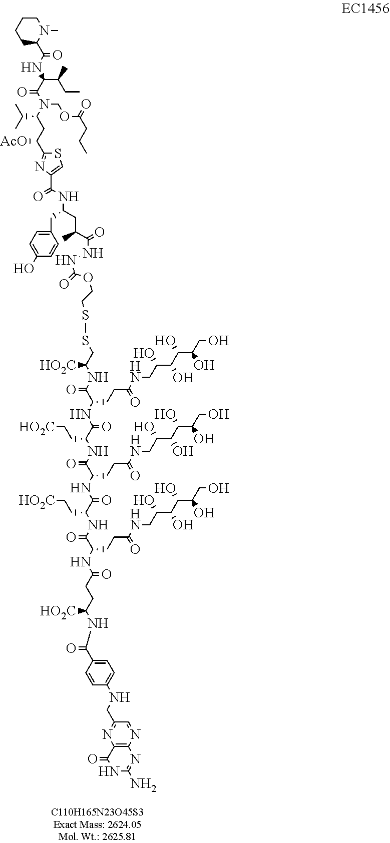 [SMILES: CCCC(=O)OCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

and related compounds, and pharmaceutically acceptable salts thereof.

A pharmaceutical composition comprising the compound of the preceding clause in combination with one or more carriers, diluents, or excipients, or a combination thereof.

A unit dose or unit dosage form composition comprising a therapeutically effective amount of the compound of any one of the preceding clauses, optionally in combination with one or more carriers, diluents, or excipients, or a combination thereof.

A composition for treating cancer in a host animal, the composition comprising comprising a therapeutically effective amount of the compound of any one of the preceding clauses; or a pharmaceutical composition comprising a therapeutically effective amount of the compound of any one of the preceding clauses, optionally further comprising one or more carriers, diluents, or excipients, or a combination thereof.

A method for treating cancer in a host animal, the method comprising the step of administering to the host animal a composition comprising a therapeutically effective amount of the compound of any one of the preceding clauses; or a pharmaceutical composition comprising the compound of any one of the preceding clauses, optionally further comprising one or more carriers, diluents, or excipients, or a combination thereof.

Use of the compound of any one of the preceding clauses, optionally in combination with one or more carriers, diluents, or excipients, or a combination thereof, in the manufacture of a medicament for treating a cancer in a host animal.

The method or composition or unit dose or use of any one of the preceding clauses wherein the cancer is drug resistant cancer.

The method or composition or unit dose or use of any one of the preceding clauses wherein the cancer is a vinca resistant cancer, such as a vinblastine and/or desacetylvinblastine monohydrazide resistant cancer.

The method or composition or unit dose or use of any one of the preceding clauses wherein the cancer is a platinum resistant cancer.

The method or composition or unit dose or use of any one of the preceding clauses wherein the cancer is a cisplatin resistant cancer.

The method or composition or unit dose or use of any one of the preceding clauses wherein the cancer is a taxol-family resistant cancer.

The method or composition or unit dose or use of any one of the preceding clauses wherein the cancer is a paclitaxel resistant cancer.

The method or composition or unit dose or use of any one of the preceding clauses wherein the cancer is an ovarian cancer.

The method or composition or unit dose or use of any one of the preceding clauses wherein the cancer is a drug resistant ovarian cancer.

The method or composition or unit dose or use of any one of the preceding clauses wherein the cancer is a cisplatin resistant ovarian cancer.

The method or composition or unit dose or use of any one of the preceding clauses wherein the cancer is a platinum resistant ovarian cancer, such as NCI/ADR-RES or NCI/ADR-RES related ovarian cancer.

The method or composition or unit dose or use of any one of the preceding clauses wherein the cancer is a platinum resistant ovarian cancer, such as IGROVCDDP or IGROVCDDP related ovarian cancer.

The method or composition or unit dose or use of any one of the preceding clauses wherein the cancer is a breast cancer.

The method or composition or unit dose or use of any one of the preceding clauses wherein the cancer is a drug resistant breast cancer.

The method or composition or unit dose or use of any one of the preceding clauses wherein the cancer is a triple negative breast cancer, such as MDA-MB-231 or MDA-MB-231 related breast cancer.

The method or composition or unit dose or use of any one of the preceding clauses wherein the cancer is a non-small cell lung cancer.

The method or composition or unit dose or use of any one of the preceding clauses wherein the cancer is a hepatocellular carcinoma or cancer.

The method or composition or unit dose or use of any one of the preceding clauses further comprising the step of administering an additional anti-cancer agent to the host animal, where the combination of the composition, and the additional anti-cancer agent is administered in a therapeutically effective amount.

The method or composition or unit dose or use of any one of the preceding clauses further comprising the step of administering one or more additional anti-cancer agents to the host animal, where the combination of the composition, and the one or more additional anti-cancer agents are administered in a therapeutically effective amount.

The method or composition or unit dose or use of any one of the preceding clauses wherein the administering step includes the composition administered at an otherwise sub-optimal therapeutic dose.

The method or composition or unit dose or use of any one of the preceding clauses wherein the administering step includes the composition administered at an otherwise less toxic dose.

The method or composition or unit dose or use of any one of the preceding clauses wherein the administering step includes the additional anti-cancer agent administered at an otherwise sub-optimal therapeutic dose.

The method or composition or unit dose or use of any one of the preceding clauses wherein the administering step includes the one or more additional anti-cancer agents administered at an otherwise sub-optimal therapeutic dose.

The method or composition or unit dose or use of any one of the preceding clauses wherein the administering step includes the additional anti-cancer agent at administered at an otherwise less toxic dose.

The method or composition or unit dose or use of any one of the preceding clauses wherein the administering step includes the one or more additional anti-cancer agents at administered at an otherwise less toxic dose.

The method or composition or unit dose or use of any one of the preceding clauses wherein the additional anti-cancer agent has a mode of action selected from the group consisting of intercalating or inhibiting macromolecular biosynthesis, inhibiting progression of the enzyme topoisomerase II, relaxing DNA supercoils, inhibiting transcription, stabilizating topoisomerase II complexes, preventing DNA double helices from being resealed, inhibiting DNAreplication, inducing histone eviction from chromatin; crosslinking DNA, eliciting DNA repair mechanisms, which in turn activate apoptosis; inhibiting angiogenesis; inhibiting topoisomerase-1; binding and/or stabilizing microtubules, preventing physiological microtubule depolymerisation/disassembly leading to apoptosis, phosphorylating oncoprotein bcl-2 leading to apoptosis unblocking, suppressing microtubule dynamic assembly and disassembly; inhibiting spindle function, suppressing microtubule dynamics, suppressing microtubule detachment from centrosomes; and interfering with DNA repair, and combinations thereof.

The method or composition or unit dose or use of any one of the preceding clauses wherein the additional anti-cancer agent has a mode of action of intercalating or inhibiting macromolecular biosynthesis, inhibiting progression of the enzyme topoisomerase II, relaxing DNA supercoils, inhibiting transcription, stabilizating topoisomerase II complexes, preventing DNA double helices from being resealed, inhibiting DNAreplication, inducing histone eviction from chromatin; crosslinking DNA, eliciting DNA repair mechanisms, which in turn activate apoptosis; inhibiting angiogenesis; inhibiting topoisomerase-1; binding and/or stabilizing microtubules, preventing physiological microtubule depolymerisation/disassembly leading to apoptosis, phosphorylating oncoprotein bcl-2 leading to apoptosis unblocking, suppressing microtubule dynamic assembly and disassembly; inhibiting spindle function, suppressing microtubule dynamics, suppressing microtubule detachment from centrosomes; and interfering with DNA repair, and combinations thereof.

The method or composition or unit dose or use of any one of the preceding clauses wherein the one or more additional anti-cancer agents has a mode of action selected from the group consisting of intercalating or inhibiting macromolecular biosynthesis, inhibiting progression of the enzyme topoisomerase II, relaxing DNA supercoils, inhibiting transcription, stabilizating topoisomerase II complexes, preventing DNA double helices from being resealed, inhibiting DNAreplication, inducing histone eviction from chromatin; crosslinking DNA, eliciting DNA repair mechanisms, which in turn activate apoptosis; inhibiting angiogenesis; inhibiting topoisomerase-1; binding and/or stabilizing microtubules, preventing physiological microtubule depolymerisation/disassembly leading to apoptosis, phosphorylating oncoprotein bcl-2 leading to apoptosis unblocking, suppressing microtubule dynamic assembly and disassembly; inhibiting spindle function, suppressing microtubule dynamics, suppressing microtubule detachment from centrosomes; and interfering with DNA repair.

The method or composition or unit dose or use of any one of the preceding clauses wherein the one or more additional anti-cancer agents has a mode of action of intercalating or inhibiting macromolecular biosynthesis, inhibiting progression of the enzyme topoisomerase II, relaxing DNA supercoils, inhibiting transcription, stabilizating topoisomerase II complexes, preventing DNA double helices from being resealed, inhibiting DNAreplication, inducing histone eviction from chromatin; crosslinking DNA, eliciting DNA repair mechanisms, which in turn activate apoptosis; inhibiting angiogenesis; inhibiting topoisomerase-1; binding and/or stabilizing microtubules, preventing physiological microtubule depolymerisation/disassembly leading to apoptosis, phosphorylating oncoprotein bcl-2 leading to apoptosis unblocking, suppressing microtubule dynamic assembly and disassembly; inhibiting spindle function, suppressing microtubule dynamics, suppressing microtubule detachment from centrosomes; and interfering with DNA repair, and combinations thereof.

The method or composition or unit dose or use of any one of the preceding clauses wherein the additional anti-cancer agent is selected from the group consisting of doxorubicin (DOXIL), cisplatin, bevacizumab (Avastin), topotecan, docetaxel, paclitaxel, and carboplatin, and pharmaceutically acceptable salts of the foregoing, and combinations thereof.

The method or composition or unit dose or use of any one of the preceding clauses wherein the additional anti-cancer agent is doxorubicin (DOXIL), or a pharmaceutically acceptable salt thereof.

The method or composition or unit dose or use of any one of the preceding clauses wherein the additional anti-cancer agent is cisplatin, or pharmaceutically acceptable salt thereof.

The method or composition or unit dose or use of any one of the preceding clauses wherein the additional anti-cancer agent is bevacizumab (Avastin), or a pharmaceutically acceptable salt thereof.

The method or composition or unit dose or use of any one of the preceding clauses wherein the additional anti-cancer agent is topotecan, or a pharmaceutically acceptable salt thereof.

The method or composition or unit dose or use of any one of the preceding clauses wherein the additional anti-cancer agent is docetaxel, or a pharmaceutically acceptable salt thereof.

The method or composition or unit dose or use of any one of the preceding clauses wherein the additional anti-cancer agent is paclitaxel, or a pharmaceutically acceptable salt thereof.

The method or composition or unit dose or use of any one of the preceding clauses wherein the additional anti-cancer agent is carboplatin, or a pharmaceutically acceptable salt thereof.

The method or composition or unit dose or use of any one of the preceding clauses wherein the one or more additional anti-cancer agents is one or more of doxorubicin (DOXIL), cisplatin, bevacizumab (Avastin), topotecan, docetaxel, paclitaxel, and carboplatin, and pharmaceutically acceptable salts of the foregoing, and combinations thereof.

The method or composition or unit dose or use of any one of the preceding clauses wherein the one or more additional anti-cancer agents is a combination of paclitaxel and carboplatin, or pharmaceutically acceptable salts thereof.

In another embodiment, the compounds described herein can be internalized into the targeted pathogenic cells by binding to the corresponding cell surface receptor. In particular, vitamin receptors, such as folate receptors, selectively and/or specifically bind the vitamin, and internalization can occur, for example, through receptor-mediated endocytosis. Once internalized, the releasable linker included in the compounds described herein allows for the delivery of the drug cargo to the interior of the target cell, thus decreasing toxicity against non-target tissues because the releasable linker remains substantially or completely intact until the compounds described herein are delivered to the target cells. Accordingly, the compounds described herein act intracellularly by delivering the drug to an intracellular biochemical process, a decrease the amount of unconjugated drug exposure to the host animal's healthy cells and tissues.

In another embodiment, compounds described herein that include a folate receptor binding ligand exhibit greater specificity for the folate receptor compared to the corresponding compounds that do not include at least one unnatural amino acid. In another embodiment, compounds described herein that include a folate receptor binding ligand show high activity for folate receptor expressing cells. In another embodiment, compounds described herein exhibit potent in vitro and in vivo activity against pathogenic cells, such as KB cells, including cisplatin resistant KB cells, NCI/ADR-RES-Cl₂ cells, IGROV1 cells, and MDA-MB-231 cells. In another embodiment, compounds described herein that include a folate receptor binding ligand do not show significant binding to folate receptor negative cells. In another embodiment, compounds described herein that include a folate receptor binding ligand enter cells preferentially or exclusively via the high affinity folate receptors, such as folate receptor alpha (α) and/or folate receptor beta (β). In another embodiment, compounds described herein generally do not substantially enter cells via passive transport, such as via the reduced folate carrier (RFC). In another embodiment, compounds described herein exhibit lower host animal toxicity compared to compounds that do not include at least one unnatural amino acid. In another embodiment, compounds described herein exhibit greater serum stability compared to compounds that do not include at least one unnatural amino acid. In another embodiment, compounds described herein are cleared rapidly compared to compounds that do not include at least one unnatural amino acid. In another embodiment, compounds described herein are cleared primarily via renal clearance compared to hepatic clearance.

The compounds described herein can be used for both human clinical medicine and veterinary applications. Thus, the host animal harboring the population of pathogenic cells and treated with the compounds described herein can be human or, in the case of veterinary applications, can be a laboratory, agricultural, domestic, or wild animal. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The cancer cell population can arise spontaneously or by such processes as mutations present in the germline of the host animal or somatic mutations, or it can be chemically-, virally-, or radiation-induced.

The invention can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, including triple negative breast, testicular, prostate, rectal, kidney, endometrial, liver and lung cancers, including non-small cell lung.

Further, the additional anti-cancer agent can be one that is cytotoxic, enhances tumor permeability, inhibits tumor cell proliferation, promotes apoptosis, decreases anti-apoptotic activity in target cells, is used to treat diseases caused by infectious agents, enhances an endogenous immune response directed to the pathogenic cells, or is useful for treating a disease state caused by any type of pathogenic cell. Additional illustrative anti-cancer agents include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, rhizoxin, T2 toxin, plant alkaloids, prednisone, hydroxyurea, teniposide, mitomycins, discodermolides, microtubule inhibitors, epothilones, tubulysins, cyclopropyl benz[e]indolone, seco-cyclopropyl benz[e]indolone, O—Ac-seco-cyclopropyl benz[e]indolone, bleomycin and any other antibiotic, nitrogen mustards, nitrosureas, vinca alkaloids, such as vincristine, vinblastine, vindesine, vinorelbine and analogs and derivative thereof such as deacetylvinblastine monohydrazide (DAVLBH), colchicine, colchicine derivatives, allocolchicine, thiocolchicine, trityl cysteine, halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other drug or toxin. Other drugs that can be included in the conjugates described herein include rapamycins, such as sirolimus or everolimus, penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, and any other antimicrobial compound.

Further, the one or more additional anti-cancer agent can be those that are cytotoxic, enhances tumor permeability, inhibits tumor cell proliferation, promotes apoptosis, decreases anti-apoptotic activity in target cells, is used to treat diseases caused by infectious agents, enhances an endogenous immune response directed to the pathogenic cells, or is useful for treating a disease state caused by any type of pathogenic cell. Additional illustrative anti-cancer agents include those described in the preceeding paragraph.

Further, the at least one additional anti-cancer agent can be one that is cytotoxic, enhances tumor permeability, inhibits tumor cell proliferation, promotes apoptosis, decreases anti-apoptotic activity in target cells, is used to treat diseases caused by infectious agents, enhances an endogenous immune response directed to the pathogenic cells, or is useful for treating a disease state caused by any type of pathogenic cell. Additional illustrative anti-cancer agents include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, rhizoxin, T2 toxin, plant alkaloids, prednisone, hydroxyurea, teniposide, mitomycins, discodermolides, microtubule inhibitors, epothilones, tubulysins, cyclopropyl benz[e]indolone, seco-cyclopropyl benz[e]indolone, O—Ac-seco-cyclopropyl benz[e]indolone, bleomycin and any other antibiotic, nitrogen mustards, nitrosureas, vinca alkaloids, such as vincristine, vinblastine, vindesine, vinorelbine and analogs and derivative thereof such as deacetylvinblastine monohydrazide (DAVLBH), colchicine, colchicine derivatives, allocolchicine, thiocolchicine, trityl cysteine, halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other drug or toxin. Other drugs that can be included in the conjugates described herein include rapamycins, such as sirolimus or everolimus, penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, and any other antimicrobial compound, and combinations thereof.

In another embodiment, the anti-cancer agent is selected from cryptophycins, bortezomib, thiobortezomib, tubulysins, aminopterin, rapamycins, paclitaxel, docetaxel, doxorubicin, daunorubicin, everolimus, α-amanatin, verucarin, didemnin B, geldanomycin, purvalanol A, ispinesib, budesonide, dasatinib, epothilones, maytansines, and tyrosine kinase inhibitors, including analogs and derivatives of the foregoing.

In another embodiment, the one or more anti-cancer agents are selected from the group consisting of one or more cryptophycins, bortezomib, thiobortezomib, tubulysins, aminopterin, rapamycins, paclitaxel, docetaxel, doxorubicin, daunorubicin, everolimus, α-amanatin, verucarin, didemnin B, eribulin mesylate (Halaven®) geldanomycin, purvalanol A, ispinesib, budesonide, dasatinib, epothilones, maytansines, and tyrosine kinase inhibitors, and combinations thereof.

The drug delivery conjugate compounds described herein can be administered in a additional combination therapies with any other known drug whether or not the additional drug is targeted. Illustrative additional drugs include, but are not limited to, peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins, antigens and antibodies thereto, haptens and antibodies thereto, hormones, lipids, phospholipids, liposomes, toxins, antibiotics, analgesics, bronchodilators, beta-blockers, antimicrobial agents, antihypertensive agents, cardiovascular agents including antiarrhythmics, cardiac glycosides, antianginals, vasodilators, central nervous system agents including stimulants, psychotropics, antimanics, and depressants, antiviral agents, antihistamines, cancer drugs including chemotherapeutic agents, tranquilizers, anti-depressants, H-2 antagonists, anticonvulsants, antinauseants, prostaglandins and prostaglandin analogs, muscle relaxants, anti-inflammatory substances, stimulants, decongestants, antiemetics, diuretics, antispasmodics, antiasthmatics, anti-Parkinson agents, expectorants, cough suppressants, mucolytics, and mineral and nutritional additives.

In another embodiment, at least one additional composition comprising a therapeutic factor can be administered to the host in combination or as an adjuvant to the above-detailed methodology, to enhance the drug delivery conjugate-mediated elimination of the population of pathogenic cells, or more than one additional therapeutic factor can be administered. The therapeutic factor can be selected from a compound capable of stimulating an endogenous immune response, a chemotherapeutic agent, or another therapeutic factor capable of complementing the efficacy of the administered drug delivery conjugate. The method of the invention can be performed by administering to the host, in addition to the above-described conjugates, compounds or compositions capable of stimulating an endogenous immune response (e.g. a cytokine) including, but not limited to, cytokines or immune cell growth factors such as interleukins 1-18, stem cell factor, basic FGF, EGF, G-CSF, GM-CSF, FLK-2 ligand, HILDA, MIP-1α, TGF-α, TGF-β, M-CSF, IFN-α, IFN-β, IFN-γ, soluble CD23, LIF, and combinations thereof.

Therapeutically effective combinations of these factors can be used. In one embodiment, for example, therapeutically effective amounts of IL-2, for example, in amounts ranging from about 0.1 MIU/m²/dose/day to about 15 MIU/m²/dose/day in a multiple dose daily regimen, and IFN-α, for example, in amounts ranging from about 0.1 MIU/m²/dose/day to about 7.5 MIU/m²/dose/day in a multiple dose daily regimen, can be used along with the drug delivery conjugates to eliminate, reduce, or neutralize pathogenic cells in a host animal harboring the pathogenic cells (MIU=million international units; m²=approximate body surface area of an average human). In another embodiment IL-12 and IFN-α are used in the above-described therapeutically effective amounts for interleukins and interferons, and in yet another embodiment IL-15 and IFN-α are used in the above described therapeutically effective amounts for interleukins and interferons. In an alternate embodiment IL-2, IFN-α or IFN-γ, and GM-CSF are used in combination in the above described therapeutically effective amounts. The invention also contemplates the use of any other effective combination of cytokines including combinations of other interleukins and interferons and colony stimulating factors.

It is understood that the linker may be neutral or ionizable under certain conditions, such as physiological conditions encountered in vivo. For ionizable linkers, under the selected conditions, the linker may deprotonate to form a negative ion, or alternatively become protonated to form a positive ion. It is appreciated that more than one deprotonation or protonation event may occur. In addition, it is understood that the same linker may deprotonate and protonate to form inner salts or zwitterionic compounds.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

As used herein the term “radical” with reference to, for example, the cell surface receptor binding and/or targeting ligand, and/or the independently selected drug, refers to a cell surface receptor binding and/or targeting ligand, and/or an independently selected drug, as described herein, where one or more atoms or groups, such as a hydrogen atom, or an alkyl group on a heteroatom, and the like, is removed to provide a radical for conjugation to the polyvalent linker L. Such ligand radicals and drug radicals may also be referred herein as ligand analogs and drug analogs, respectively.

As used herein, the term “leaving group” refers to a reactive functional group that generates an electrophilic site on the atom to which it is attached such that nucleophiles may be added to the electrophilic site on the atom. Illustrative leaving groups include, but are not limited to, halogens, optionally substituted phenols, acyloxy groups, sulfonoxy groups, and the like. It is to be understood that such leaving groups may be on alkyl, acyl, and the like. Such leaving groups may also be referred to herein as activating groups, such as when the leaving group is present on acyl. In addition, conventional peptide, amide, and ester coupling agents, such as but not limited to PyBop, BOP-Cl, BOP, pentafluorophenol, isobutylchloroformate, and the like, form various intermediates that include a leaving group, as defined herein, on a carbonyl group.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is appreciated that certain functional groups, such as the hydroxy, amino, and like groups form complexes and/or coordination compounds with water and/or various solvents, in the various physical forms of the compounds. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like.

Illustrative routes for parenteral administration include intravenous, intraarterial, intraperitoneal, epidural, intraurethral, intrasternal, intramuscular and subcutaneous, as well as any other art recognized route of parenteral administration.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

The compounds described herein may be prepared using conventional processes, including those described in International Patent Publication Nos. WO 2009/002993, WO 2004/069159, WO 2007/022494, and WO 2006/012527, and U.S. patent application Ser. No. 13/837,539. The disclosures of each of the foregoing are herein incorporated by reference in their entirety.

Each publication cited herein is incorporated herein by reference.

The following examples further illustrate specific embodiments of the invention; however, the following illustrative examples should not be interpreted in any way to limit the invention.

### EXAMPLES

### Compound Examples

The compounds described herein may be prepared using the process and syntheses described herein, as well as using general organic synthetic methods. In particular, methods for preparing the compounds are described in U.S. patent application publication 2005/0002942, the disclosure of which is incorporated herein by reference.

### Example

General formation of folate-peptides. The folate-containing peptidyl fragment Pte-Glu-(AA)ₙ-NH(CHR₂)CO₂H (3) is prepared by a polymer-supported sequential approach using standard methods, such as the Fmoc-strategy on an acid-sensitive Fmoc-AA-Wang resin (1), as shown in the following Scheme:

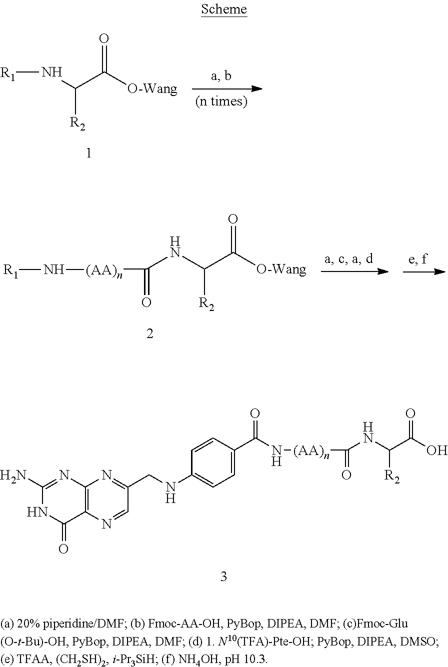

It is to be understood that unnatural amino acids may be included in the foregoing process using the appropriate starting materials.

In this illustrative embodiment of the processes described herein, R₁ is Fmoc, R₂ is the desired appropriately-protected amino acid side chain, and DIPEA is diisopropylethylamine. Standard coupling procedures, such as PyBOP and others described herein or known in the art are used, where the coupling agent is illustratively applied as the activating reagent to ensure efficient coupling. Fmoc protecting groups are removed after each coupling step under standard conditions, such as upon treatment with piperidine, tetrabutylammonium fluoride (TBAF), and the like. Appropriately protected amino acid building blocks, such as Fmoc-Glu-OtBu, Fmoc-D-Glu-OtBu, N¹⁰-TFA-Pte-OH, and the like, are used, as described in the Scheme, and represented in step (b) by Fmoc-AA-OH. Thus, AA refers to any amino acid starting material, that is appropriately protected. It is to be understood that the term amino acid as used herein is intended to refer to any reagent having both an amine and a carboxylic acid functional group separated by one or more carbons, and includes the naturally occurring alpha and beta amino acids, as well as amino acid derivatives and analogs of these amino acids. In particular, amino acids having side chains that are protected, such as protected serine, threonine, cysteine, aspartate, and the like may also be used in the folate-peptide synthesis described herein. Further, gamma, delta, or longer homologous amino acids may also be included as starting materials in the folate-peptide synthesis described herein. Further, amino acid analogs having homologous side chains, or alternate branching structures, such as norleucine, isovaline, β-methyl threonine, β-methyl cysteine, β,β-dimethyl cysteine, and the like, may also be included as starting materials in the folate-peptide synthesis described herein.

The coupling sequence (steps (a) & (b)) involving Fmoc-AA-OH is performed “n” times to prepare solid-support peptide (2), where n is an integer and may equal 0 to about 100. Following the last coupling step, the remaining Fmoc group is removed (step (a)), and the peptide is sequentially coupled to a glutamate derivative (step (c)), deprotected, and coupled to TEA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups that may form part of the appropriately-protected amino acid side chain. The TEA protecting group is removed upon treatment with base (step (f)) to provide the folate-containing peptidyl fragment (3).

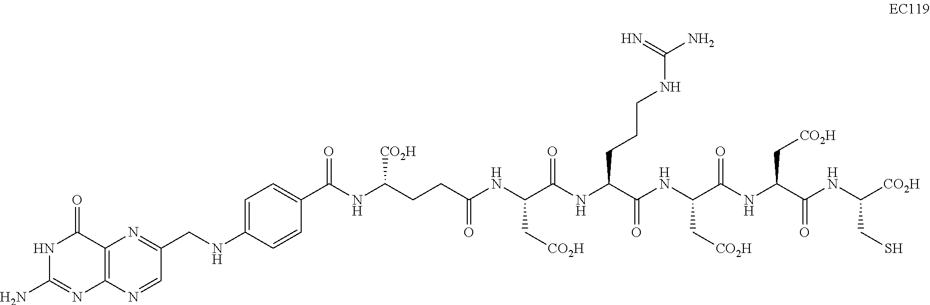 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

LCMS [ESI [M+H]⁺: 1046; Partial ¹H NMR (D₂O, 300 MHz): δ 8.68 (s, 1H, FA H-7), 7.57 (d, 2H, J=8.4 Hz, FA H-12 &16), 6.67 (d, 2H, J=9 Hz, FA H-13 &15), 4.40-4.75 (series of m, 5H), 4.35 (m, 2H), 4.16 (m, 1H), 3.02 (m, 2H), 2.55-2.95 (series of m, 8H), 2.42 (m, 2H), 2.00-2.30 (m, 2H), 1.55-1.90 (m, 2H), 1.48 (m, 2H) ppm.

### Example

### Preparation of Tubulysin Hydrazides. Illustrated by Preparing EC0347 (TubB-H)

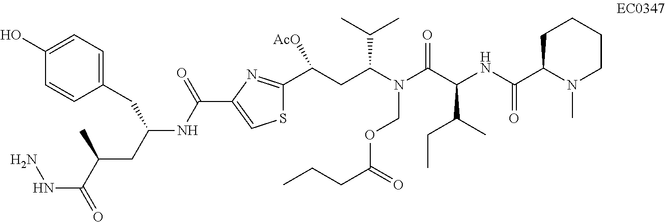 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NN)cs1)C(C)C]

N,N-Diisopropylethylamine (DIPEA, 6.1 μL) and isobutyl chloroformate (3.0 μL) were added with via syringe in tandem into a solution of tubulysin B (0.15 mg) in anhydrous EtOAc (2.0 mL) at −15° C. After stirring for 45 minutes at −15° C. under argon, the reaction mixture was cooled down to −20° C. and to which was added anhydrous hydrazine (5.0 μL). The reaction mixture was stirred under argon at −20° C. for 3 hours, quenched with 1.0 mM sodium phosphate buffer (pH 7.0, 1.0 mL), and injected into a preparative HPLC for purification. Column: Waters XTerra Prep MS C₁₈ 10 μm, 19×250 mm; Mobile phase A: 1.0 mM sodium phosphate buffer, pH 7.0; Mobile phase B: acetonitrile; Method: 10% B to 80% B over 20 minutes, flow rate=25 mL/min. Fractions from 15.14-15.54 minutes were collected and lyophilized to produce EC0347 as a white solid (2.7 mg). The foregoing method is equally applicable for preparing other tubulysin hydrazides by the appropriate selection of the tubulysin starting compound.

### Example

### Synthesis of Coupling Reagent EC0311

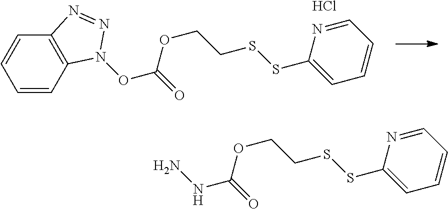 [SMILES: Cl ; O=C(OCCSSc1ccccn1)On1nnc2ccccc21 ; NNC(=O)OCCSSc1ccccn1]

DIPEA (0.60 mL) was added to a suspension of HOBt-OCO₂—(CH₂)₂—SS-2-pyridine HCl (685 mg, 91%) in anhydrous DCM (5.0 mL) at 0° C., stirred under argon for 2 minutes, and to which was added anhydrous hydrazine (0.10 mL). The reaction mixture was stirred under argon at 0° C. for 10 minutes and room temperature for an additional 30 minutes, filtered, and the filtrate was purified by flash chromatography (silica gel, 2% MeOH in DCM) to afford EC0311 as a clear thick oil (371 mg), solidified upon standing.

### Example

### Preparation of Tubulysin Disulfides (Stepwise Process)

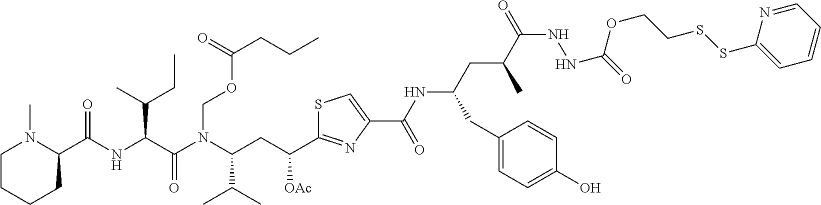 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ccccn2)cs1)C(C)C]

Illustrated for EC0312. DIPEA (36 μL) and isobutyl chloroformate (13 μL) were added by syringe in tandem into a solution of tubulysin B (82 mg) in anhydrous EtOAc (2.0 mL) at −15° C. After stirring for 45 minutes at −15° C. under argon, to the reaction mixture was added a solution of EC0311 in anhydrous EtOAc (1.0 mL). The resulting solution was stirred under argon at −15° C. for 15 minutes and room temperature for an additional 45 minutes, concentrated, and the residue was purified by flash chromatography (silica gel, 2 to 8% MeOH in DCM) to give EC0312 as a white solid (98 mg). The foregoing method is equally applicable for preparing other tubulysin derivatives by the appropriate selection of the tubulysin starting compound.

### Example

### Tubulysin B Pyridyldisulfide

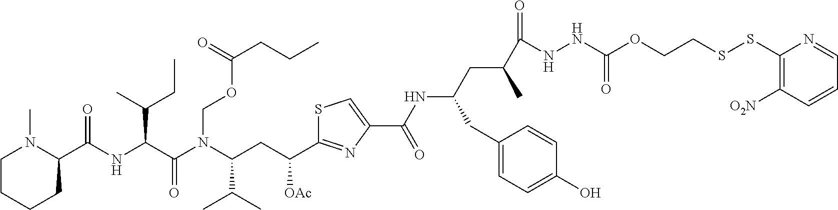 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ncccc2[N+](=O)[O-])cs1)C(C)C]

Similarly, Tubulysin B pyridyldisulfide is prepared as described herein.

### Example

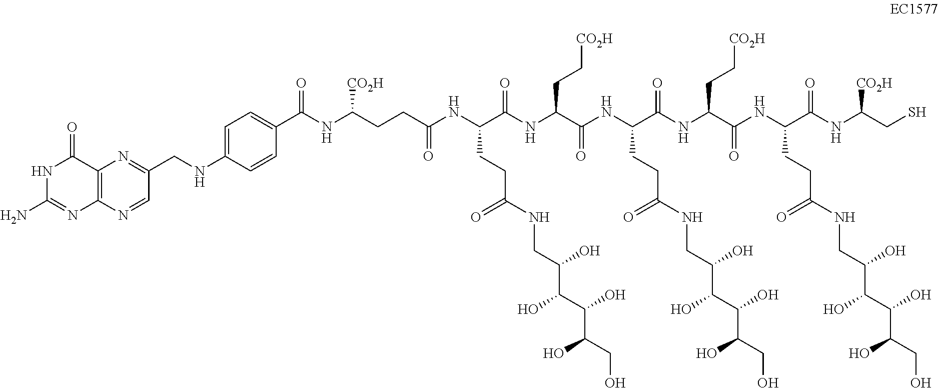 [SMILES: Nc1nc2ncc(CNc3ccc(C(=O)N[C@@H](CCC(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@H](CS)C(=O)O)C(=O)O)cc3)nc2c(=O)[nH]1]

MS (ESI, [M+H]⁺)=1681. Partial ¹H NMR (D₂O): 8.96 (s), 7.65 (d), 6.81 (d), 4.66 (s), 4.40-4.15 (m), 3.90-3.54 (m), 3.50-3.18 (m), 2.97-2.90 (m), 2.51-1.80 (m).

### Example

### General Synthesis of Disulfide Containing Tubulysin Conjugates

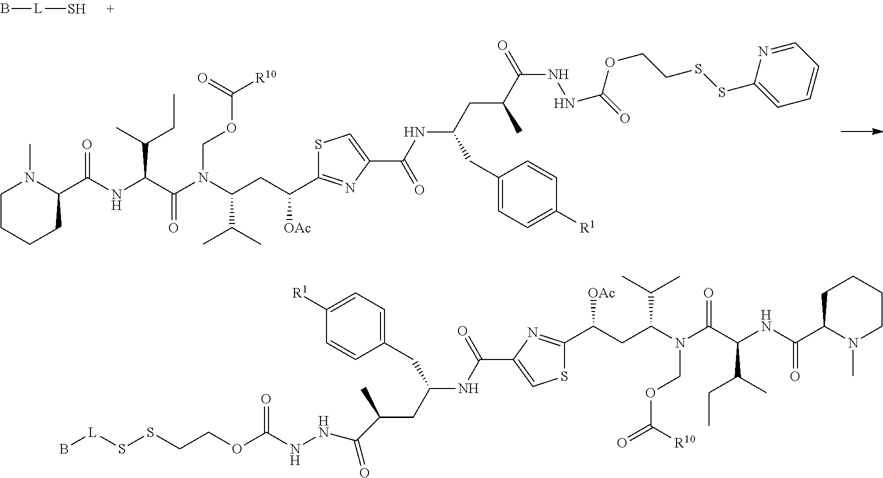

Illustrated with pyridinyl disulfide derivatives of certain naturally occurring tubulysins, where R¹ is H or OH, and R¹⁰, is alkyl or alkenyl. A binding ligand-linker intermediate containing a thiol group is taken in deionized water (ca. 20 mg/mL, bubbled with argon for 10 minutes prior to use) and the pH of the suspension was adjusted by saturated NaHCO₃ (bubbled with argon for 10 minutes prior to use) to about 6.9 (the suspension may become a solution when the pH increased). Additional deionized water is added (ca. 20-25%) to the solution as needed, and to the aqueous solution is added immediately a solution of EC0312 in THF (ca. 20 mg/mL). The reaction mixture becomes homogenous quickly. After stirring under argon, e.g. for 45 minutes, the reaction mixture is diluted with 2.0 mM sodium phosphate buffer (pH 7.0, ca 150 volume percent) and the THF is removed by evacuation. The resulting suspension is filtered and the filtrate may be purified by preparative HPLC (as described herein). Fraction are lyophilized to isolate the conjugates. The foregoing method is equally applicable for preparing other tubulysin conjugates by the appropriate selection of the tubulysin starting compound.

### Example

EC1456 and its preparation are described in WO2014/062697 at pages 76 to 91, which pages are incorporated herein by reference. EC1456 is prepared according to the following process.

### Example

EC1426 is prepared according to the following process.

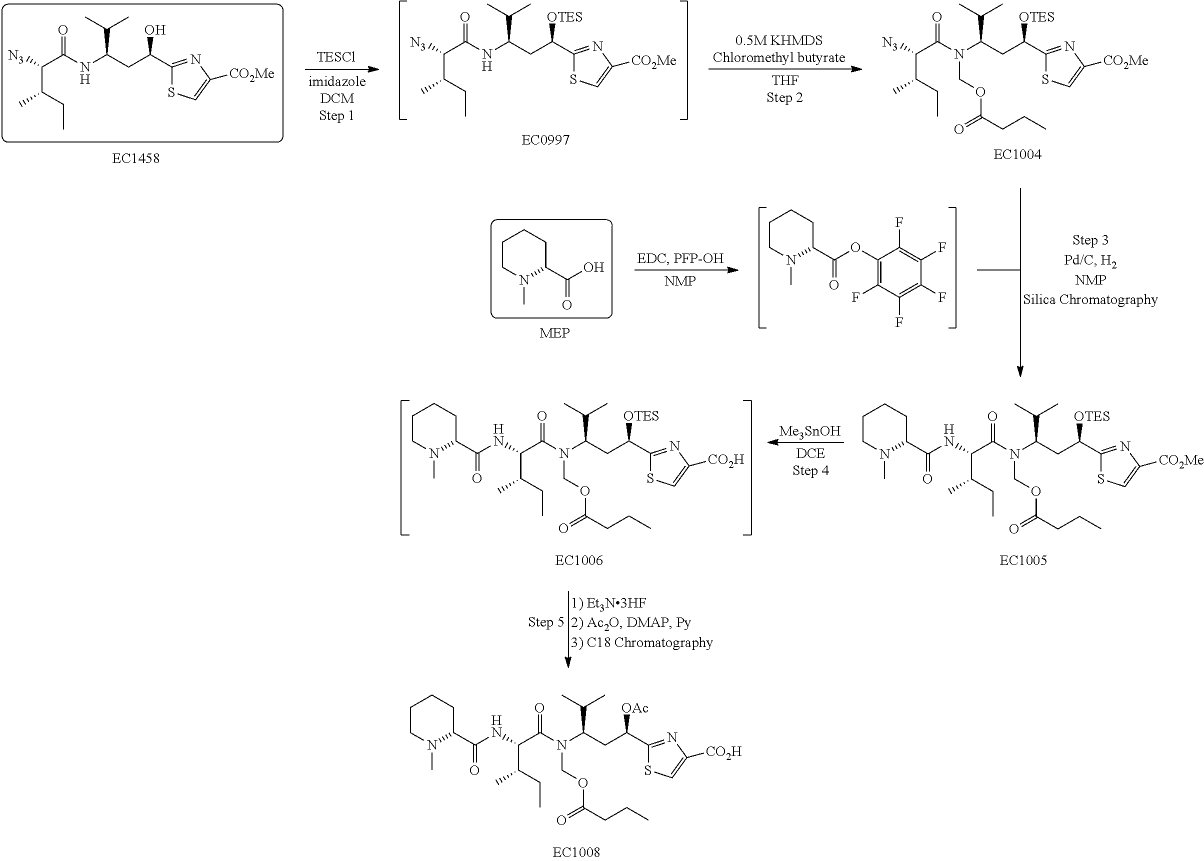

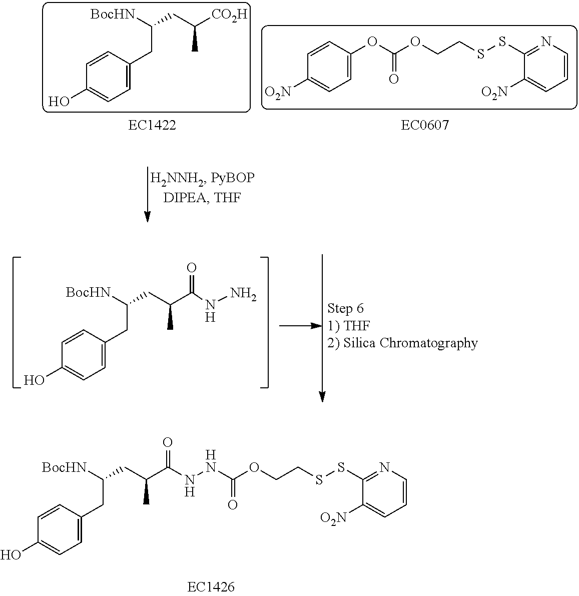

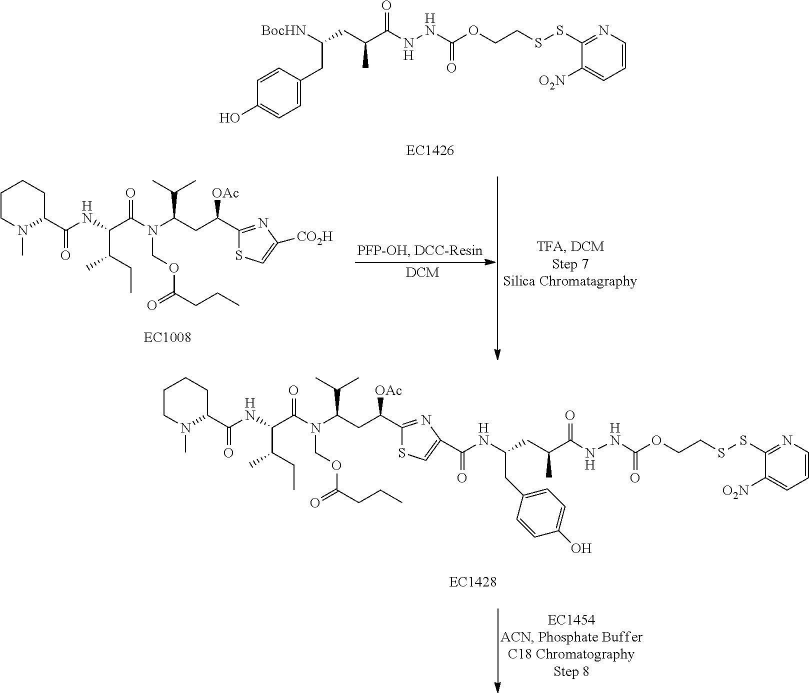

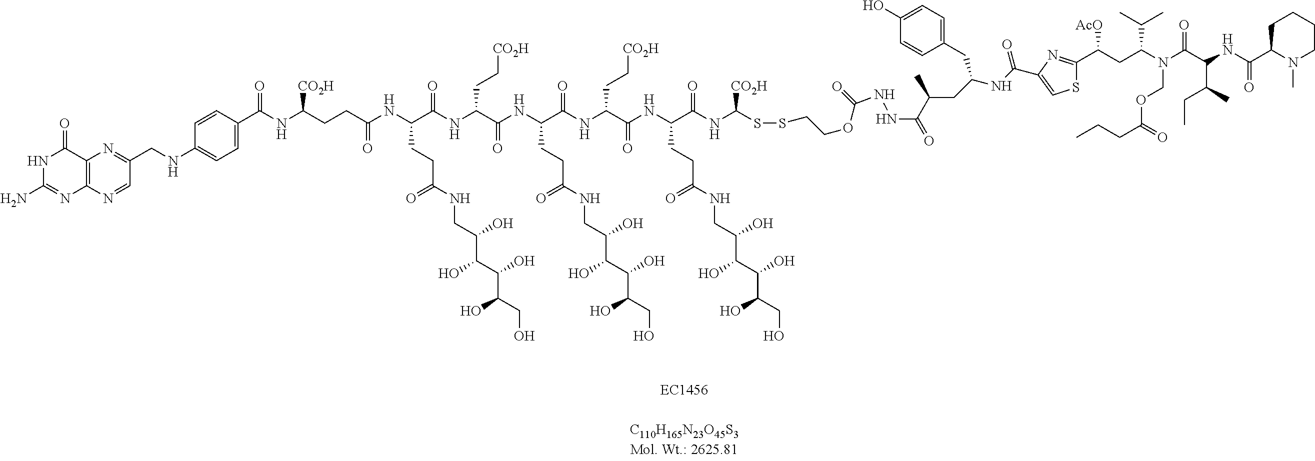

### Example

Additional tubulsyins and tubulysin intermediates may be prepared according to the processes described in WO 2012/019123, WO 2009/055562, PCT International Application Serial No. U52013/034672, and U.S. Provisional application Ser. No. 61/793,082, the disclosures of each of which are incorporated herein by reference in their entirety.

### Example

EC1454 is prepared according to the following process.

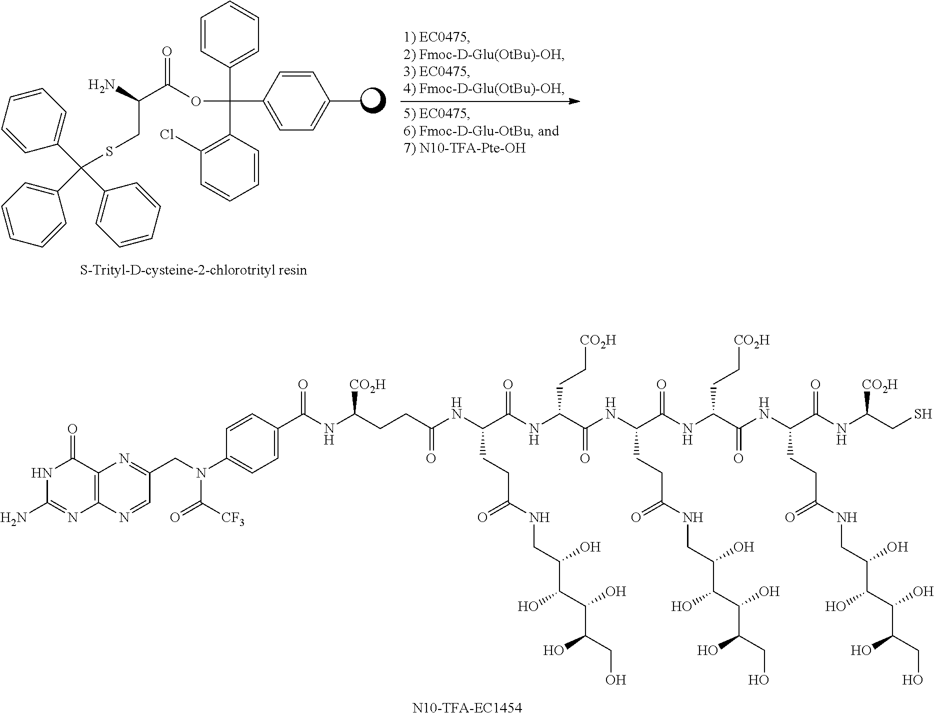 [SMILES: Cc1ccc(C(OC(=O)[C@H](N)CSC(c2ccccc2)(c2ccccc2)c2ccccc2)(c2ccccc2)c2ccccc2Cl)cc1 ; Nc1nc2ncc(CN(C(=O)C(F)(F)F)c3ccc(C(=O)N[C@H](CCC(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CS)C(=O)O)C(=O)O)cc3)nc2c(=O)[nH]1]

The solid phase synthesis of N¹⁰-TFA protected EC1454 starts with resin bound trityl protected D-cysteine. The resin is suspended in dimethylformamide (DMF) and washed twice with DMF. EC0475 (glucamine modified L-glutamic acid), (Benzotriazol-1-yloxy)tripyrrolidinophosphonium hexafluorophosphate (PyBOP), and diisopropylethylamine (DIPEA) are added to reaction mixture. After at least 1 hour, a Kaiser test is performed to ensure the coupling is complete. The resin is washed three times with DMF, three times with IPA, and three times with DMF. The resin is slowly washed three times with piperidine in DMF, three times with DMF, and three times with IPA. A Kaiser test is performed to confirm deprotection. The resin is washed three times with DMF and the next amino acid in the sequence is coupled following the same process. Monomers are coupled in the following order: 1) EC0475, 2) Fmoc-D-Glu(OtBu)-OH, 3) EC0475, 4) Fmoc-D-Glu(OtBu)-OH, 5) EC0475, 6) Fmoc-D-Glu-OtBu, and 7) N¹⁰-TFA-Pte-OH.

Once the final coupling is complete, the resin is washed three times with methanol and dried by passing argon through the resin at room temperature. The dried resin is suspended in a mixture of TFA, water, ethanedithiol, and triisopropylsilane. After 1 hour the resin is removed by filtration and washed with TEA. The product is precipitated by addition to cold ethyl ether, filtered, and washed with ether. The solids are dried under vacuum at room temperature and stored in a freezer.

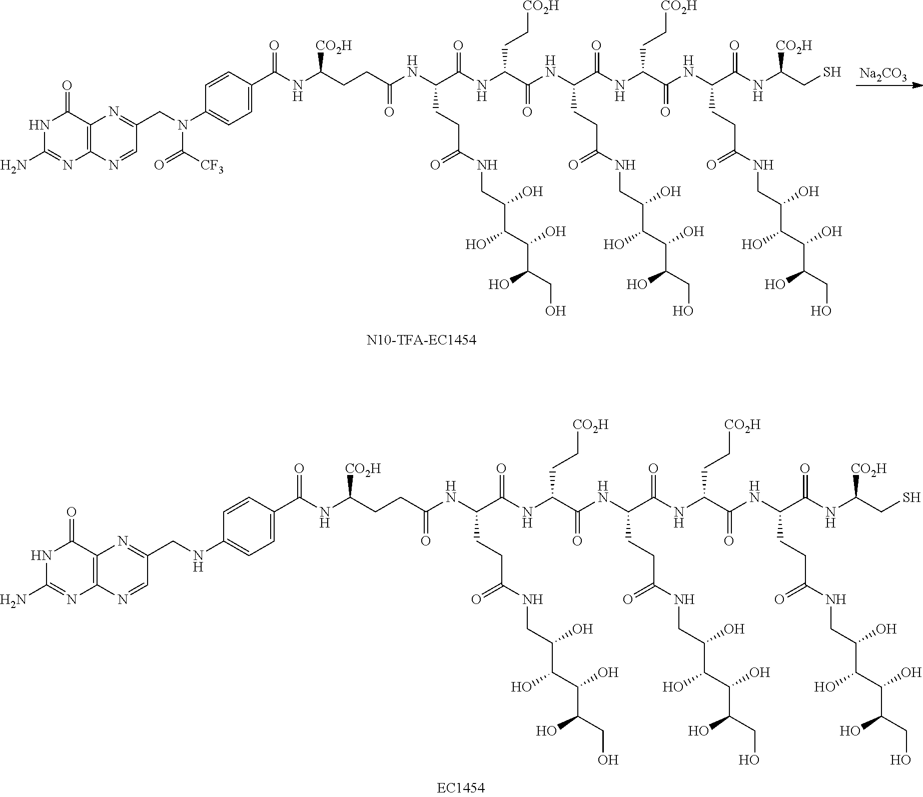

N¹⁰-TFA EC1454 is dissolved in argon sparged water. Sodium carbonate (1M in water, argon sparged) is added to achieve a pH of 9.4-10.1. The reaction mixture is stirred for at least 20 minutes. Once the reaction is complete as determined by LC, it is quenched by adjusting the pH to 1.9-2.3 with 2M HCl. The product is purified by C18 column chromatography using acetonitrile and pH 5 ammonium acetate buffer as eluents. Fractions are collected and checked for purity by HPLC. The combined product fractions are concentrated on a rotary evaporator and then lyophilized to yield EC1454 as a yellow solid. MS (ESI, [M+2H]²⁺)=840.90, [M+H1]⁺=1681.3. Selected 1H-NMR (DMSO, 300 MHz) δ(ppm): 8.6 (s), 7.6 (d), 6.6 (d), 4.45 (s), 4.35 (t), 4.15-4.3 (m), 3.3-3.6 (m), 3.25 (m), 3.0 (m), 2.7-2.9 (m), 2-2.3 (m), 1.6-2 (m). The product is stored at −20° C.

### Example

EC1456 is prepared according to the following process.

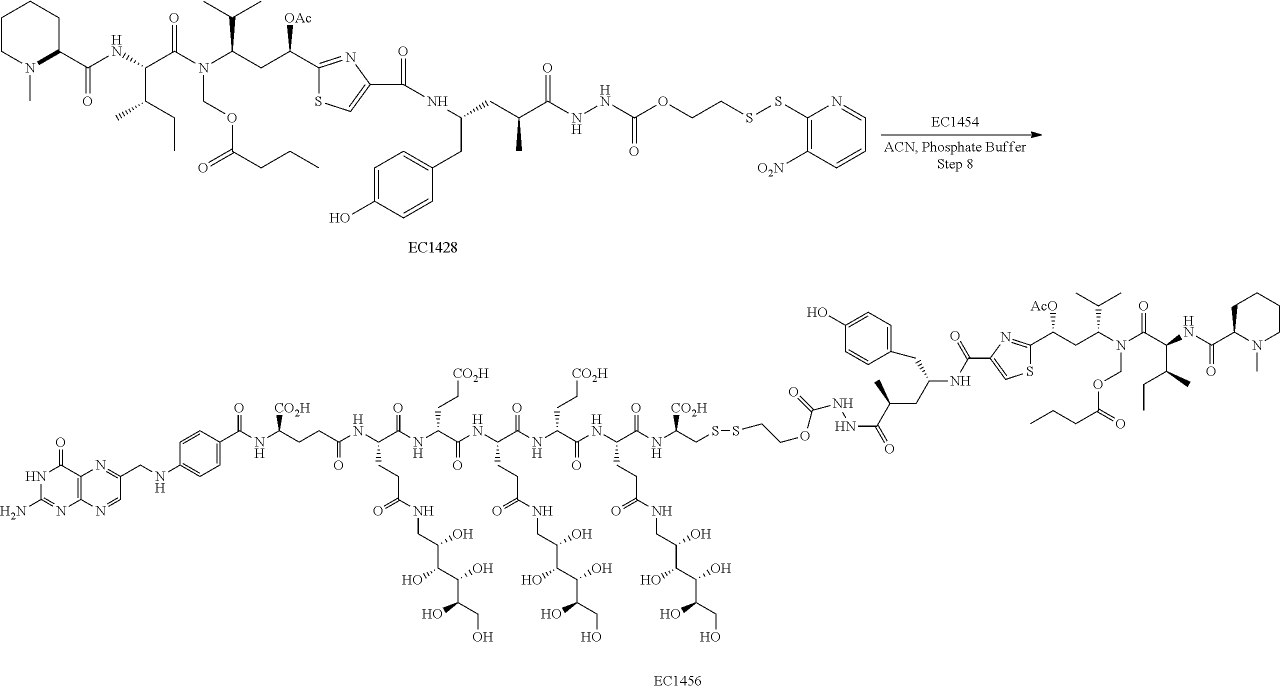

EC1428 is dissolved in acetonitrile and a solution of EC1454 in pH 7.4 Sodium phosphate buffer is added. The solutions are sparged with argon before and after addition. The reaction mixture is stirred for at least 15 minutes and then checked for completion. The desired product is purified by C18 column chromatography using acetonitrile and pH 7.4 phosphate buffer as eluents. The product fractions are collected, checked for purity, combined and concentrated by ultra-filtration to yield an aqueous solution that is 10-20 mg/mL EC1456. The final product solution is sampled for assay and then stored in a freezer.

The positive electrospray mass spectrum of EC1456 was obtained on a high resolution Waters Acquity UPLC Xevo Gs-S QTOF mass spectrometer. The spectrum was obtained following separation of the major component on a UPLC inlet system, the resolving power was approximately 35,000. The accurate mass measurement of the M+H monoisotopic peak was 2625.0598, which is 1.1 ppm error difference from the theoretical value of 2625.0570 for an ion of formula C₁₁₀H₁₆₆N₂₃O₄₅S₃. The isotopic distribution is also consistent with that formula.

Mass spectral features of the ES+ spectrum for EC1456

| Observed | |
|---|---|
| Ion | Interpretation |
| 2626.06 | ¹³C isotope of the (M + H)⁺ ion for the MW 2624 drug |
| | substance |
| 1313.54 | ¹³C isotope of the (M + 2H)⁺⁺ ion for the MW 2624 drug |
| | substance |
| 1150.43 | ¹³C isotope of the (M + 2H − 326)⁺⁺ fragment, |
| | corresponding to the cleavage of the peptide bond at the |
| | tertiary nitrogen and the loss of the butyric acid moiety. |
| 876.03 | ¹³C isotope of the (M + 3H)⁺⁺⁺ ion for the MW 2624 drug |
| | substance |
| 657.27 | ¹³C isotope of the (M + 4H)⁺⁺⁺⁺ ion for the MW 2624 drug |
| | substance |

A sample of ˜30 mg EC1456 was dissolved in 665 μL of a 9:1 mixture of deuterated dimethylsulfoxide and deuterated water. The ¹H NMR spectrum was obtained at 500 MHz at 26 deg. C. on an Agilent model DD2 spectrometer fitted with a 2 channel probe containing both broadband and proton observe coils. The ¹³C NMR spectrum was obtained at 125 MHz on the same instrument under identical conditions. All spectra were referenced to the DMSO solvent residual signals at 2.5 ppm (¹H) and 39.50 ppm (¹³C).

All spectral features are assigned for both NMR spectra in the tables below (¹H and ¹³C) using the atom numbering in the following figure, where the * symbols indicate the connection for the disulfide bond.

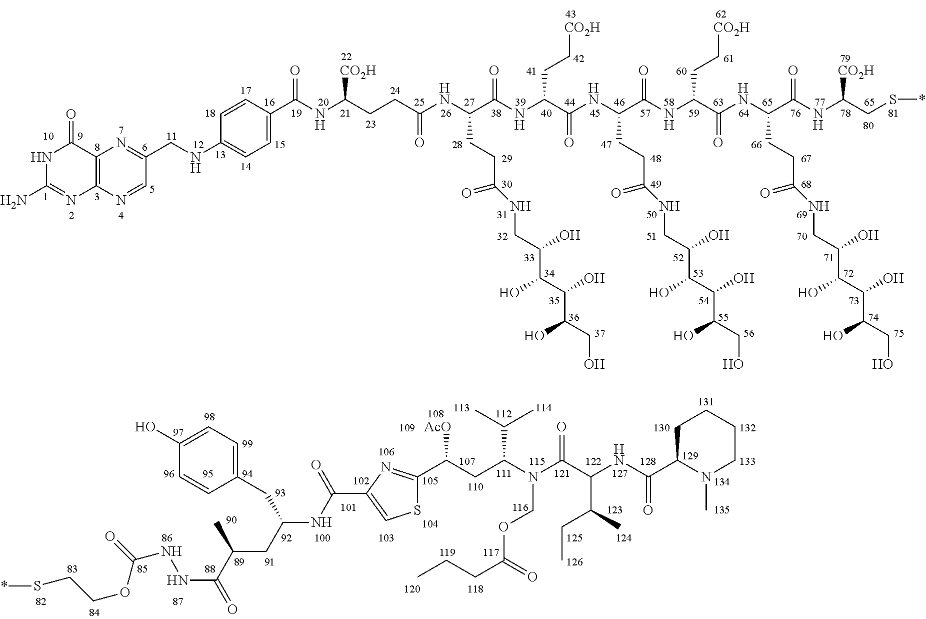 [SMILES: *SC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c1ccc(NCc2cnc3nc(N)[nH]c(=O)c3n2)cc1)C(=O)O)C(=O)O ; *SCCOC(=O)NNC(=O)[C@@H](C)C[C@H](Cc1ccc(O)cc1)NC(=O)c1csc([C@@H](C[C@H](C(C)C)N(COC(=O)CCC)C(=O)C(NC(=O)[C@H]2CCCCN2C)[C@@H](C)CC)OC(C)=O)n1]

Assignments were made on the basis of both 1D and 2D NMR experiments, including through bond H—H connectivity using the COSY and TCSY 2D experiments, through space H—H proximity using 2D NOESY, carbon multiplicity measurement using the 1D DEPT experiment and through bond C—H connectivity using the proton detected 2D experiments HSQC and HMBC. In most cases of overlap in the 1D spectra (different protons or carbons resonating at the same chemical shift) could be resolved in the 2D spectra, in these cases the tables reflect the chemical shifts measured from the 2D spectra but summed integrations for the group of co-resonating species. In some cases of 1D overlap (such as the nearly identical glutamic acid and glucamine subunits) there was also overlap in the 2D correlation spectra which precludes unambiguous assignment of single or multiple resonances between multiple atom numbers, in these cases there are multiple entries for chemical shift and/or atom number assignments in a single table row.

NH and OH protons were exchanged by the D₂O deuterium atoms and are mostly absent from the spectrum, except weak broad peaks in the 5-10 ppm region. The ¹H peaks in the spectrum that are not listed in the table include a broad HOD peak at 3.75 ppm, and a DMSO peak at 2.50 ppm. The HOD peak does not obscure any resonances, but elevates the integrations for nearby resonances at 4.2 and 3.4-3.7 ppm due to the broad baseline rise. The DMSO peak obscures the resonance for H129, which is not integrated for this reason. The ¹³C peaks in spectrum not listed in the table include the very large DMSO solvent at 39.50 ppm. The DMSO peak obscures both the signals from C91 and C93. The C116 peak is not observable in the ¹³C spectrum due to extensive broadening due to conformational changes around the nearby amide group. All three chemical shifts (C91, C93, C116) are visible in and measured in the proton detected 2D correlation spectra.

### Proton NMR Assignments for EC1456

| Proton | | |
|---|---|---|
| Chemical Shift (ppm) | Assignment | # protons |
| 8.61 | 5 | 1 |
| 8.16 | 103 | 1 |
| 7.58 | 15, 17 | 2 |
| 6.96 | 95, 99 | 2 |
| 6.62 | 14, 18 | 4 |
| 6.59 | 96, 98 | |
| 6.18 | 116 Ha | 1 |
| 5.7 | 107 | 1 |
| 5.24 | 116 Hb | 1 |
| 4.47 | 11 | 2 |
| 4.39 | 111, 122 | 2 |
| 4.21 | 78 | 10 |
| 4.21 | 65 | |
| 4.18 | 84 | |
| 4.15 | 46 | |
| 4.15 | 59 | |
| 4.13 | 21 | |
| 4.13 | 40 | |
| 4.09 | 27 | |
| 4.09 | 92 | |
| 3.61 | 33, 52, 71 | 3 |
| 3.56 | 34, 53, 72 | 6 |
| 3.54 | 37Ha, 56Ha, 75Ha | |
| 3.46 | 36, 55, 74 | 3 |
| 3.4 | 35, 54, 73 | 6 |
| 3.38 | 37Hb, 56Hb, 75Hb | |
| 3.21 | 80Ha, 32Ha, 51 Ha, | 4 |
| | 70 Ha | |
| 3.05 | 32Hb, 51Hb, 70Hb | 3 |
| 2.93 | 80 Hb | 3 |
| 2.91 | 83 | |
| 2.8 | 133Ha | 1 |
| 2.68 | 93 | 2 |
| 2.49 (see text) | 129 | 1 |
| 2.35 | 89 | 2 |
| 2.33 | 110Ha | |
| 2.8 | 133Hb | 37 |
| 2.17 | 118 | |
| 2.14-2.08 | 24, 29, 42, 48, 61, 67 | |
| 2.09 | 110Hb | |
| 2.08 | 109 | |
| 2.02 | 135 | |
| 1.97-1.70 | 28, 41, 47, 60, 66 | |
| 1.92 | 23Ha | |
| 1.88 | 123 | |
| 1.8 | 91Ha | |
| 1.79 | 23Hb | |
| 1.77 | 112 | |
| 1.6 | 131Ha | 9 |
| 1.56 | 130Ha | |
| 1.5 | 132Ha | |
| 1.5 | 91Hb | |
| 1.45 | 125Ha | |
| 1.42 | 119 | |
| 1.4 | 132Hb | |
| 1.33 | 130Hb | |
| 1.14 | 131Hb | 2 |
| 1.07 | 125Hb | |
| 1 | 90 | 3 |
| 0.94 | 114 | 3 |
| 0.79 | 124 | 3 |
| 0.77 | 126 | 3 |
| 0.75 | 120 | 3 |
| 0.64 | 113 | 3 |

### Carbon NMR Assignments for EC1456

| Carbon | |
|---|---|
| Chemical shift | |
| (ppm) | Assignment |
| 176.77, 176.32 | 43, 62 |
| 175.74 | 88 |
| 175.42 | 22 |
| 174.75 | 121 |
| 173.87, 172.68, | 25, 38, 44, 57, 63 |
| 172.15, 171.94, | |
| 171.84 | |
| 173.43 | 79 |
| 173.3 | 128 |
| 172.79 (2x), | 30, 49, 68 |
| 172.72 | |
| 172.46 | 117 |
| 170.87 | 76 |
| 170.39 | 108 |
| 169.3 | 105 |
| 166.09 | 19 |
| 162.4 | 9 |
| 160.7 | 101 |
| 156.4 | 85 |
| 156.09 | 3 |
| 155.71 | 97 |
| 154.59 | 1 |
| 150.84 | 13 |
| 149.63 | 102 |
| 149.11 | 6 |
| 148.99 | 5 |
| 130.44 | 95, 99 |
| 128.99 | 15, 17 |
| 128.89 | 94 |
| 127.99 | 8 |
| 124.97 | 103 |
| 122.24 | 16 |
| 115.25 | 96, 98 |
| 111.86 | 14, 18 |
| 72.17 (3x) | 35, 54, 73 |
| 71.78, 71.74, 71.71 | 33, 52, 71 |
| 71.62, 71.59 (2x) | 36, 55, 74 |
| 69.65, 69.57 (2x) | 34, 53, 72 |
| 69.45 | 107 |
| 69.34 | 116 |
| 68.51 | 129 |
| 63.42 (3x) | 37, 56, 75 |
| 63.03 | 84 |
| 55.08 | 133 |
| 54.05 | 40 |
| 53.88 | 78 |
| 53.46 (2x) | 46, 59 |
| 53.33 | 27 |
| 52.96 (2x) | 122, 111 |
| 52.89 | 21 |
| 52.55 | 65 |
| 49.77 | 92 |
| 46.07 | 11 |
| 44.02 | 135 |
| 42.85 | 80 |
| 42.34 (2x), 42.29 | 32, 51, 70 |
| 39.52 | 93 |
| 38.95 | 91 |
| 37.43 | 83 |
| 35.95 | 118 |
| 35.43 | 123 |
| 35.38 | 89 |
| 34.86 | 110 |
| 32.56, 32.36, | 24, 29, 42, 48, 61, 67 |
| 32.16, 32.09 (2x), | |
| 31.81 | |
| 30.5 | 112 |
| 29.95 | 130 |
| 28.60, 28.04, 27.78 | 28, 41, 47, 60, 66 |
| (2x), 27.66 | |
| 27 | 23 |
| 25.01 | 132 |
| 24.43 | 125 |
| 23.04 | 131 |
| 20.86 | 109 |
| 20.56 | 114 |
| 19.64 | 113 |
| 18.36 | 90 |
| 18.04 | 119 |
| 15.64 | 124 |
| 13.72 | 120 |
| 10.28 | 126 |

The IR spectrum of EC1456 was acquired on a Nexus 6700® Fourier transform infrared (FT-IR) spectrophotometer (Thermo Nicolet) equipped with an Ever-Glo mid/far IR source, an extended range potassium bromide (KBr) beam splitter, and a deuterated triglycine sulfate (DTGS) detector. An attenuated total reflectance (ATR) accessory (Thunderdome™, Thermo Spectra-Tech), with a germanium (Ge) crystal was used for data acquisition. The spectrum represents 256 co-added scans collected at a spectral resolution of 4 cm-1. A background data set was acquired with a clean Ge crystal. A Log 1/R (R=reflectance) spectrum was acquired by taking a ratio of these two data sets against each other. Wavelength calibration was performed using polystyrene.

Infrared Band Assignments for EC1456 Reference Substance

| Characteristic Absorption(s) (cm⁻¹) | Functional Group |
|---|---|
| 1700-1500 (m, m) | Aromatic C═C Bending |
| 2950-2850 (m or s) | Alkyl C—H Stretch |
| ~3030 (v) | Aromatic C—H Stretch |
| 3550-3200 (broad, s) | Alcohol/Phenol O—H Stretch |
| 3700-3500 (m) | Amide C═O Stretch |

The ultraviolet spectrum EC1456 acquired on a Perkin-Elmer Lambda 25 UV/Vis spectrometer. The spectrum was recorded at 40.7 uM in 0.1M NaOH solvent on a 1 cm path-length cell at 25 deg. C. The local maxima at 366 nm, 288 nm and 243 nm are due primarily to the Pteroic acid, benzamide/phenol and thiazole-amide substructures, respectively, although the molecule contains dozens of chromaphores with overlapping absorption in the UV region.

### Example

The following additional compounds are described and are prepared according to the general processes described herein.

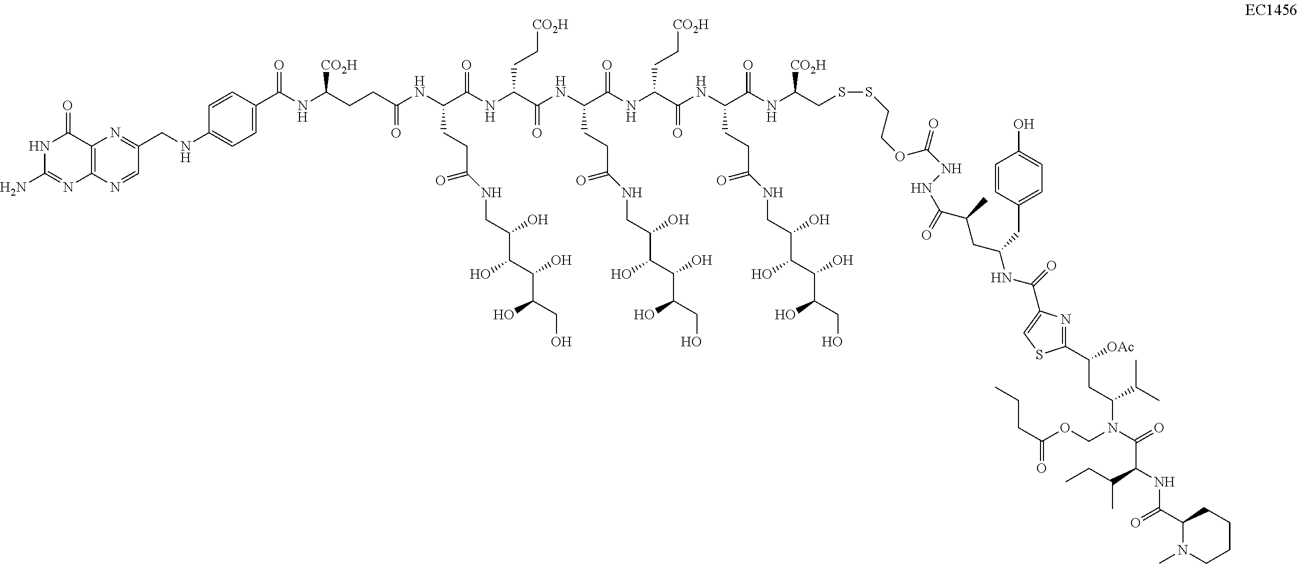 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

### Comparative Example

### EC0923

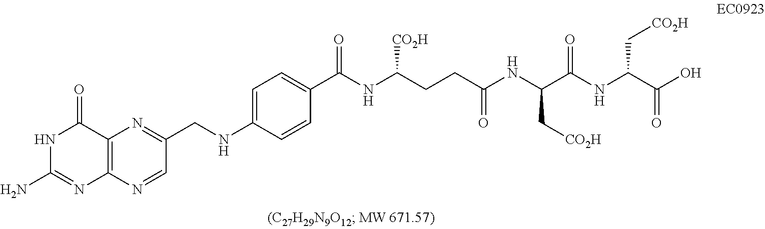 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

### Methods and Examples

General. The following abbreviations are used herein: partial response (PR); complete response (CR), three times per week (M/W/F) TIW).

METHOD. Relative Affinity Assay. The affinity for folate receptors (FRs) relative to folate were determined according to a previously described method (Westerhof, G. R., J. H. Schornagel, et al. (1995) Mol. Pharm. 48: 459-471) with slight modification. Briefly, FR-positive KB cells were heavily seeded into 24-well cell culture plates and allowed to adhere to the plastic for 18 h. Spent incubation media was replaced in designated wells with folate-free RPMI (FFRPMI) supplemented with 100 nM ³H-folic acid in the absence and presence of increasing concentrations of test article or folic acid. Cells were incubated for 60 min at 37° C. and then rinsed 3 times with PBS, pH 7.4. Five hundred microliters of 1% SDS in PBS, pH 7.4, was added per well. Cell lysates were then collected and added to individual vials containing 5 mL of scintillation cocktail, and then counted for radioactivity. Negative control tubes contained only the ³H-folic acid in FFRPMI (no competitor). Positive control tubes contained a final concentration of 1 mM folic acid, and CPMs measured in these samples (representing non-specific binding of label) were subtracted from all samples. Relative affinities are defined as the inverse molar ratio of compound required to displace 50% of ³H-folic acid bound to the FR on KB cells, where the relative affinity of folic acid for the FR is set to 1.

METHOD. Inhibition of Cellular DNA Synthesis. The compounds described herein were evaluated using an in vitro cytotoxicity assay that predicts the ability of the drug to inhibit the growth of folate receptor-positive cells, such as KB cells, RAW264.7 macrophages, and the like. It is to be understood that the choice of cell type can made on the basis of the susceptibility of those selected cells to the drug that forms the conjugate. The test compounds were comprised of folate linked to a respective chemotherapeutic drug, as prepared according to the processes described herein. The test cells were exposed to varying concentrations of folate-drug conjugate, and also in the absence or presence of at least a 100-fold excess of folic acid to assess activity as being specific to folate receptor mediation.

### Example

Conjugates of cytotoxic drugs described herein are active against KB cells. The activity is mediated by the folate receptor as indicated by competition experiments using co-administered folic acid. KB cells were exposed for up to 7 h at 37° C. to the indicated concentrations of folate-drug conjugate in the absence or presence of at least a 100-fold excess of folic acid. The cells were then rinsed once with fresh culture medium and incubated in fresh culture medium for 72 hours at 37° C. Cell viability was assessed using a ³H-thymidine incorporation assay. For compounds described herein, dose-dependent cytotoxicity was generally measurable, and in most cases, the IC₅₀ values (concentration of drug conjugate required to reduce ³H-thymidine incorporation into newly synthesized DNA by 50%) are in the low nanomolar range. Though without being bound by theory, when the cytotoxicities of the conjugates were reduced in the presence of excess free folic acid, it is believed herein that such results indicate that the observed cell death is mediated by binding to the folate receptor.

METHOD. In vitro activity against various cancer cell lines. IC50 values were generated for various cell lines. Cells were heavily seeded in 24-well Falcon plates and allowed to form nearly confluent monolayers overnight. Thirty minutes prior to the addition of the test compound, spent medium was aspirated from all wells and replaced with fresh folate-deficient RPMI medium (FFRPMI). A subset of wells were designated to receive media containing 100 μM folic acid. The cells in the designated wells were used to determine the targeting specificity. Without being bound by theory it is believed herein that the cytotoxic activity produced by test compounds in the presence of excess folic acid, i.e. where there is competition for FR binding, corresponds to the portion of the total activity that is unrelated to FR-specific delivery. Following one rinse with 1 mL of fresh FFRPMI containing 10% heat-inactivated fetal calf serum, each well received 1 mL of medium containing increasing concentrations of test compound (4 wells per sample) in the presence or absence of 100 μM free folic acid as indicated. Treated cells were pulsed for 2 h at 37° C., rinsed 4 times with 0.5 mL of media, and then chased in 1 mL of fresh medium up to 70 h. Spent medium was aspirated from all wells and replaced with fresh medium containing 5 μCi/mL ³H-thymidine. Following a further 2 h 37° C. incubation, cells were washed 3 times with 0.5 mL of PBS and then treated with 0.5 mL of ice-cold 5% trichloroacetic acid per well. After 15 min, the trichloroacetic acid was aspirated and the cell material solubilized by the addition of 0.5 mL of 0.25 N sodium hydroxide for 15 min. A 450 μL aliquot of each solubilized sample was transferred to a scintillation vial containing 3 mL of Ecolume scintillation cocktail and then counted in a liquid scintillation counter. Final results are expressed as the percentage of ³H-thymidine incorporation relative to untreated controls.

### Example

Compounds described herein exhibited potent in vitro activity against pathogenic cells, such as KB cells. Compounds described herein exhibited greater specificity for the folate receptor compared to compounds that do not include at least one unnatural amino acid. For Example, EC1456 exhibited ca. 1000-fold specificity for the folate receptor as determined by folic acid competition (specificity=difference in IC₅₀ between competed group and non-competed group), and a 4-fold improvement in specificity compared to comparator compound EC0531, which does not include a linker L having an unnatural amino acid.

### Example

Selectivity for folate receptor expressing cells. Compounds described herein showed high activity for folate receptor expressing cells. Compounds described herein did not show significant binding to folate receptor negative cells. EC1456 showed high competable binding to low and high FR expressing cells (FR+), and did not show binding to cells that do not express FR (FR−).

### Activity of EC1456 in (FR+) and (FR−) Cell Lines

| | | FR | Activity | Competable |
|---|---|---|---|---|
| Cell Line | | Expression | (IC₅₀) | up to 100 nM |
| KB | Human Cervical Carcinoma | +++ | 2.3 nM | Yes |
| NCl/ADR-RES-Cl₂ | Human ovarian Carcinoma | ++ | 1.4 nM | Yes |
| IGROV1 | Human ovarian | + | 0.72 nM | Yes |
| | adenocarcinoma | | | |
| MDA-MB-231 | Human breast | + | 0.47 nM | Yes |
| | adenocarcinoma (triple | | | |
| | negative) | | | |
| A549 | Human Lung Carcinoma | − | Inactive (a) | NA |
| H23 | Human Lung | − | Inactive | NA |
| | adenocarcinoma | | | |
| HepG2 | Human hepatocellular | − | Inactive | NA |
| | Carcinoma | | | |
| AN3CA | Human endometrial | − | Inactive | NA |
| | adenocarcinoma | | | |
| LNCaP | Human prostate | − | ~850 nM | NA |
| | adenocarcinoma | | | |

(a) activity was evaluated from 0.1-100 nM against these specifically selected (FR−) cell lines (A549, H23, HepG2, AN3CA, LNCaP); NA=not applicable.

METHOD. Inhibition of Tumor Growth in Mice. Four to seven week-old mice (Balb/c or nu/nu strains) were purchased from Harlan Sprague Dawley, Inc. (Indianapolis, Ind.). Normal rodent chow contains a high concentration of folic acid (6 mg/kg chow); accordingly, test animals were maintained on a folate-free diet (Harlan diet #TD00434) for about 1 week before tumor implantation to achieve serum folate concentrations close to the range of normal human serum, and during the Method. For tumor cell inoculation, 1×10⁶ M109 cells (a syngeneic lung carcinoma) in Balb/c strain, or 1×10⁶ KB cells in nu/nu strain, in 100 μL were injected in the subcutis of the dorsal medial area (right axilla). Tumors were measured in two perpendicular directions every 2-3 days using a caliper, and their volumes were calculated as 0.5×L×W², where L=measurement of longest axis in mm and W=measurement of axis perpendicular to L in mm. Log cell kill (LCK) and treated over control (T/C) values were then calculated according to published procedures (see, e.g., Lee et al., “BMS-247550: a novel epothilone analog with a mode of action similar to paclitaxel but possessing superior antitumor efficacy” Clin Cancer Res 7:1429-1437 (2001); Rose, “Taxol-based combination chemotherapy and other in vivo preclinical antitumor studies” J Natl Cancer Inst Monogr 47-53 (1993)).

Dosing was initiated when the s.c. tumors had an average volume between 50-100 mm³ (t₀), typically 8 days post tumor inoculation (PTI) for KB tumors, and 11 days PTI for M109 tumors. Test animals (5/group) ere injected i.v., generally three times a week TIW), for 3 weeks with varying doses, such as with 1 μmol/kg to 5 μmol/kg, of the drug delivery conjugate or with an equivalent dose volume of PBS (control), unless otherwise indicated. Dosing solutions were prepared fresh each day in PBS and administered through the lateral tail vein of the mice.

METHOD. General 4T-1 Tumor Assay. Six to seven week-old mice (female Balb/c strain) were obtained from Harlan, Inc., Indianapolis, Ind. The mice were maintained on Harlan's folate-free chow for a total of three weeks prior to the onset of and during the method. Folate receptor-negative 4T-1 tumor cells (1×10⁶ cells per animal) were inoculated in the subcutis of the right axilla. Approximately 5 days post tumor inoculation when the 4T-1 tumor average volume is 100 mm³ (t₀), mice (5/group) were injected i.v. three times a week TIW), for 3 weeks with varying doses, such as 3 μmol/kg, of drug delivery conjugate or with an equivalent dose volume of PBS (control), unless otherwise indicated herein. Tumor growth was measured using calipers at 2-day or 3-day intervals in each treatment group. Tumor volumes were calculated using the equation V=a×b²/2, where “a” is the length of the tumor and “b” is the width expressed in millimeters.

METHOD. Drug Toxicity. Persistent drug toxicity was assessed by collecting blood via cardiac puncture and submitting the serum for independent analysis of blood urea nitrogen (BUN), creatinine, total protein, AST-SGOT, ALT-SGPT plus a standard hematological cell panel at Ani-Lytics, Inc. (Gaithersburg, Md.). In addition, histopathologic evaluation of formalin-fixed heart, lungs, liver, spleen, kidney, intestine, skeletal muscle and bone (tibia/fibula) was conducted by board-certified pathologists at Animal Reference Pathology Laboratories (ARUP; Salt Lake City, Utah).

METHOD. Toxicity as Measured by Weight Loss. The percentage weight change of the test animals was determined on selected days post-tumor inoculation (PTI), and during dosing. The results were graphed.

### Example

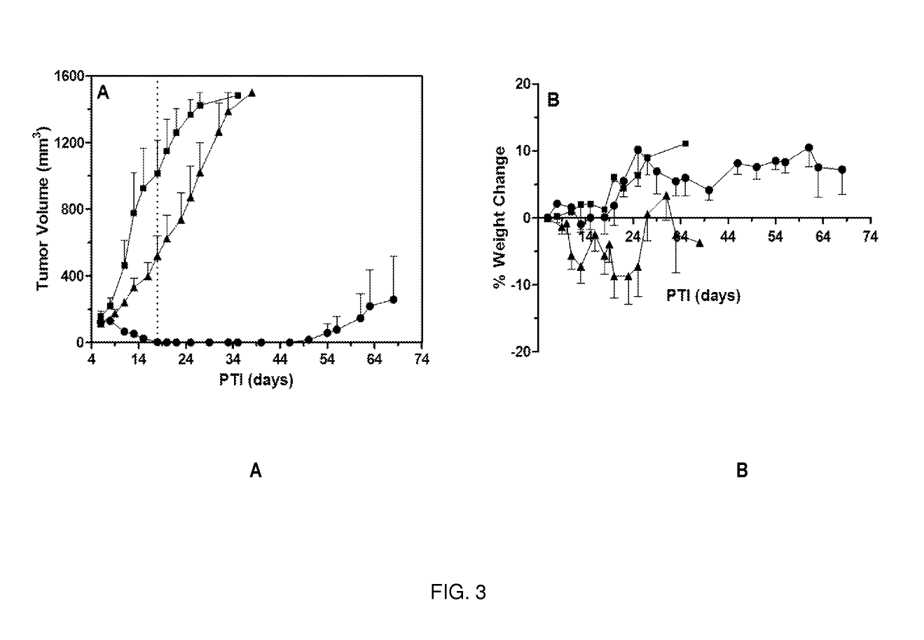

In vivo activity against tumors. Compounds described herein showed high potency and efficacy against KB tumors in nu/nu mice. Compounds described herein showed specific activity against folate receptor expressing tumors, with low host animal toxicity. For example, EC1456 showed a complete response in 4/4 test animals when administered intravenously at 1 μmol/kg TIW, 2 wk. EC1456 also showed specific activity mediated by the folate receptor as evidenced by being competable with excess comparator compound EC0923 (50 or 100 μmol/kg), as shown in FIG. 3A. EC1456 did not show any evidence of whole animal toxicity, as shown in FIG. 3B.

METHOD. TNBC Tumor Assay. Triple negative breast cancer (TNBC) is a subtype characterized by lack of gene expression for estrogen, progesterone and Her2/neu. TNBC is difficult to treat, and the resulting death rate in patients is reportedly disproportionately higher than for any other subtype of breast cancer. A TNBC xenograft model was generated in an analogous way to the KB and M109 models described herein by implanting MDA-MB-231 breast cancer cells in nu/nu mice. Dosing was initiated when the s.c. tumors have an average volume between 110-150 (generally 130) mm³ (t₀), typically 17 days post tumor inoculation (PTI). Test animals (5/group) were injected i.v., generally three times a week (TIW), for 2-3 weeks with varying doses, such as with 1 μmol/kg to 5 μmol/kg, of the drug delivery conjugate or with an equivalent dose volume of PBS (control), unless otherwise indicated. Dosing solutions were prepared fresh each day in PBS and administered through the lateral tail vein of the mice.

### Example

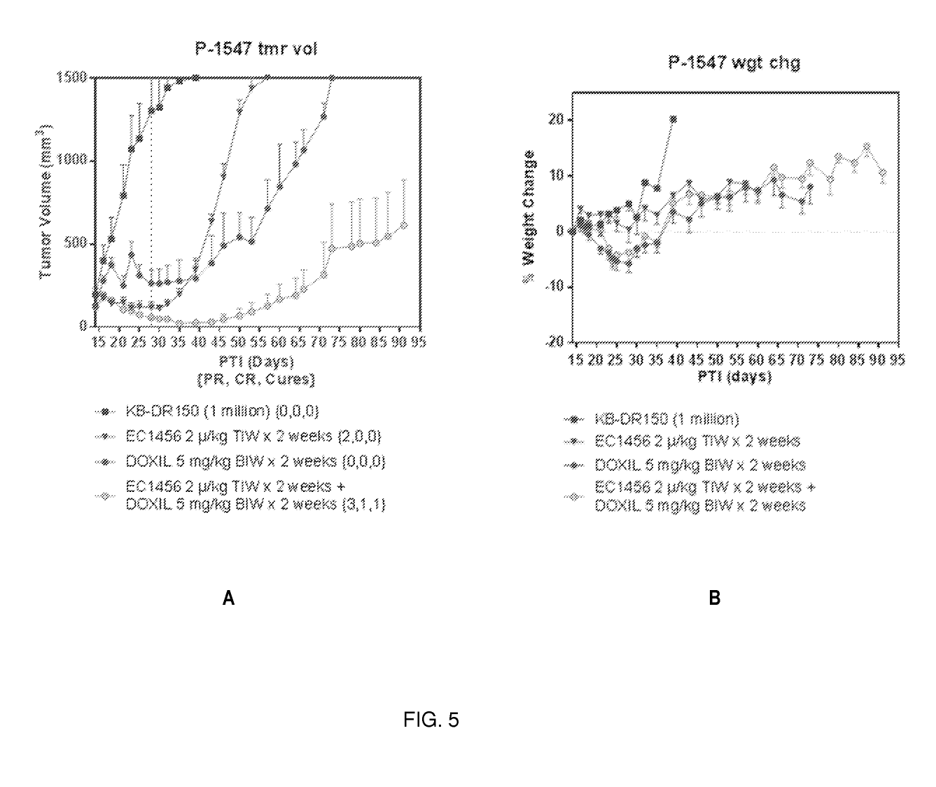

When tested against an established triple negative FR-positive subcutaneous MDA-MB-231 breast cancer xenografts, EC1456 was found to be highly active at 2 μmol/kg intravenous dose administered on a three times per week, 2 consecutive week schedule. The treatment produced 4 of 5 complete responses, where tumor volume was reduced to zero, and regrowth did not occur during the observation window over nearly 135 days. Without being bound by theory, it is believed herein that the test animals were cured of the triple negative breast cancer. The results for EC1456 are shown in FIG. 5A. The anti-tumor activity was not accompanied by significant weight loss in the test animals, as shown in FIG. 5B.

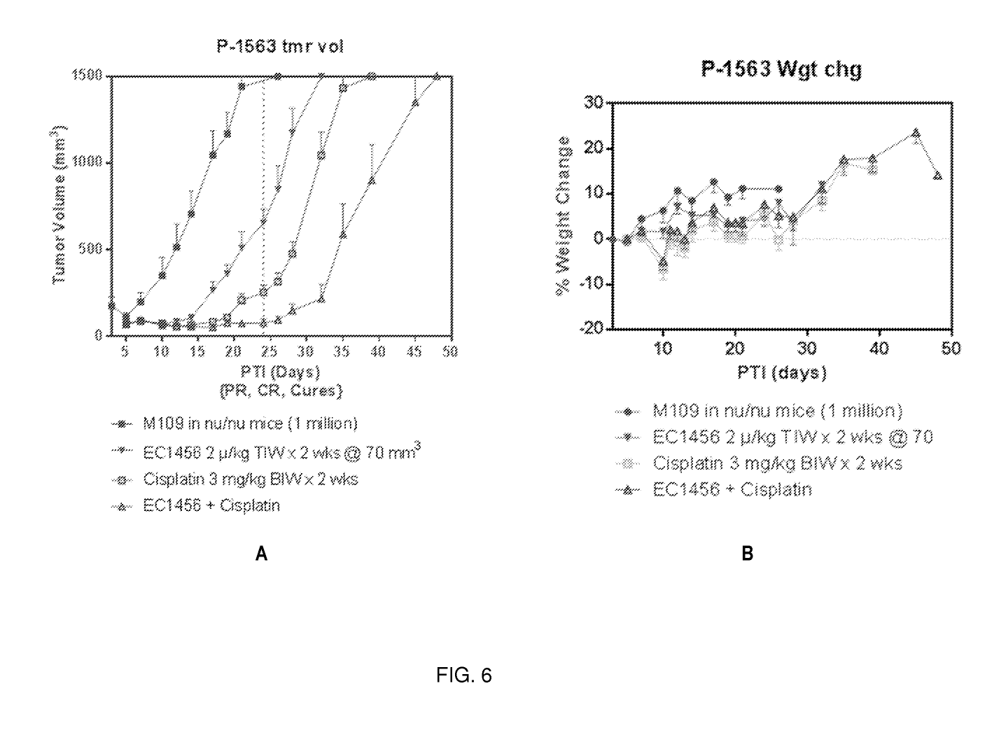

METHOD. Human cisplatin-resistant cell line. A human cisplatin-resistant cell line was created by culturing FR-positive KB cells in the presence of increasing cisplatin concentrations (100→2000 nM; over a >12 month period). The cisplatin-resistant cells, labeled as KB-CR2000 cells, were found to be tumorigenic, and were found to retain their FR expression status in vivo. KB-CR2000 tumors were confirmed to be resistant to cisplatin therapy. Treatment with a high, toxic dose of cisplatin (average weight loss of 10.3%, as shown in FIG. 6B), did not produce even a single partial response (PR), as shown in FIG. 6A. In contrast, EC1456 was found to be very active against KB-CR tumors, where 5/5 CRs were observed. In addition, regrowth of the tumor was only observed in 1/5 test animals. Without being bound by theory, it is believed herein that 4/5 test animals were cured of the cisplatin-resistant cancer, where regrowth did not occur during the nearly 70 day observation period. Furthermore, unlike cisplatin, EC1456 did not cause any weight loss in this cohort of mice, and therefore did not display any evidence of gross animal toxicity during the dosing period.

### Example

Comparison of conjugated and unconjugated drugs. The therapeutic performance of unconjugated tubulysin B and unconjugated TubB-H (EC0347) drugs was evaluated in vivo against human KB tumors in mice. The anti-tumor efficacy and gross toxicity, as determined by body weight changes, of each unconjugated drug were compared to the EC1456 conjugate. EC1456 produced dose responsive anti-tumor activity in this model. Complete responses were observed under treatment conditions that produced little to no weight loss. In contrast, both unconjugated tubulysin-based drugs failed to yield any anti-tumor response, even when very toxic doses were administered to the mice. The results are shown in the following table.

| | | | | | | Toxicity | |
|---|---|---|---|---|---|---|---|
| | Dose | Dosing | PR | CR | Cures | Deaths | Avg. Weight |
| Example | (μmol/kg) | Schedule | (%) | (%) | (%) | (%) | Loss |
| EC1456 | 0.5 | TIW, 3 weeks | 60 | 0 | 0 | 0 | <5%* |
| | 0.67 | TIW, 2 weeks | 60 | 20 | 0 | 0 | <2% |
| | 1.0 | TIW, 2 weeks | 40 | 60 | 60 | 0 | <1.5% |
| | 2.0 | TIW, 2 weeks | 0 | 100 | 100 | 0 | <3% |
| Tubulysin B | 0.1 (4 doses) | TIW, 2 weeks | 0 | 0 | 0 | 100 | >20% |
| | 0.2 (3 doses) | TIW, 2 weeks | 0 | 0 | 0 | 100 | >18% |
| | 0.5 (1 dose) | TIW, 2 weeks | 0 | 0 | 0 | 100 | >15% |
| TubB-H | 0.5 | TIW, 2 weeks | 0 | 0 | 0 | 0 | <5.5% |
| | 0.75 | TIW, 2 weeks | 0 | 0 | 0 | 20 | >10% |
| | 1.0 (2 doses)¹ | TIW, 2 weeks | 0 | 0 | 0 | 20 | >15% |

*Untreated control group had an average weight loss of 2.4%
¹Group received only 2 doses due to toxicity.

These results confirm that despite tubulysin B and TubBH being highly cytotoxic to cells in culture (typical IC₅₀˜1 nM), both agents yielded dose-limiting toxicities in mice at levels that did not produce measurable anti-tumor effect. Thus, the unconjugated compounds did not exhibit a therapeutic window. In contrast, the conjugated forms of the drugs, such as conjugated TubBH (EC1456) produced anti-tumor responses without significant toxicity to mice bearing well-established human tumor xenografts. Conjugation as described herein provided a therapeutic window to highly toxic drugs.

### Example

Compounds described herein exhibited high folate receptor affinity compared to folic acid (relative affinity=1) in 10% serum/FDRPMI, potent in vitro activity, potent in vivo activity, specificity for the folate receptor, and a sufficiently high therapeutic index compared to unconjugated drug.

| | | In | 50% | | | |
|---|---|---|---|---|---|---|
| | | vitro | com- | | | Therapeutic |
| | Relative | IC50 | petition | In vitro | In vivo | index over |
| | Affinity | (nM) | (nM) | specificity | activity | unconjugated |
| Example | (a) | (b) | (c) | (fold) (d) | (e) | drug (f) |
| EC1456 | 0.27 | 1.5 | 1416 | 944 | CR | Yes |

(a) compared to folic acid; (b) as determined by thymidine incorporation; (c) IC50 for test compound when competed with excess folic acid; the higher the IC50 the more specific is the folate mediation; (d) in vitro specificity=difference in IC₅₀ between competed group and non-competed group; (e) as determined in subcutaneous KB tumor in nu/nu mice; CR=complete response, where tumor volume, as defined herein, during the observation period was zero for all test animals in the group; (f) parent tubulysin.

METHOD. Human serum stability. Compounds described herein were tested in human serum for stability using conventional protocols and methods. The test compound was administered to the test animal, such as by subcutaneous injection. The plasma concentration of the conjugate, and optionally one or more metabolites, was monitored over time. The results were graphed to determine Cmax, Tmax, half-life, and AUC for the test compound and metabolites.

### Example

Maximum tolerated dose (MTD). Conjugate compounds described herein that include a linker comprising at least one unnatural amino acid show high MTDs, which were improved over compounds that did not have linkers comprising one or more unnatural amino acids. Test compounds were administered by i.v., BIW, 2 wks in female Sprague-Dawley rats. Comparator compound EC0531 has a MTD of 0.33 μmol/kg, while EC1456 had a MTD of at least 0.51 μmol/kg, a 65% improvement, as shown in FIG. 20. Histopathologic changes were not observed with doses of EC1456 at or below the MTD.

### Example

### Host Animals

A. Animal Receipt and Acclimation Period. Female Balb/c-derived nu/nu mice were received in good health from Harlan Laboratories (Indianapolis, Ind.).

B. Animal Housing. Upon arrival, the mice were housed at the Lilly Animal House within Purdue University located in LSA. They were immediately placed in sterilized individual ventilated cages (IVC) (polycarbonate) with bed-o-cob bedding. The cages were placed in industrial stainless steel IVC units. Five animals were assigned to a cage. Sterilized cages were replaced every 2 weeks by a qualified technician.

C. Diet and Drinking Water. Upon arrival, mice were put on irradiated Test Diet #01014 produced by Harlan Teklad, Madison, Wis. throughout the study, autoclaved RO water via water bottle was used as the drinking water. The diet and drinking water were provided ad libitum throughout the study period. One week after dosing mice were switched to Teklad Global 18% Rodent Diet (#2018S) manufactured by Harlan Teklad, Madison, Wis.

D. Environmental Conditions. All animals were housed throughout the study period in an environmentally controlled room. The room temperature settings ranged from 67° F. to 77° F. The relative humidity of the room ranged from 30% to 70%. Light timers were set to provide a 12-hour light/12-hour dark photoperiod.

E. Observations. The animals were observed daily for health.

### Example

### In Vivo Evaluation of Test Compounds

Tumor implantation. KB tumor cells were grown in folate-deficient RPMI 1640 with 5% FBS at 37° C. in a 5% CO2 humidified atmosphere. KB (1×106 cells per animal) tumor cells were inoculated subcutaneously 3 days post start of the folate deficient diet. Mice were dosed after the tumors reached between 100-150 mm3.

Preparation of dosing drug solutions. Dosing solutions were prepared when dosing began by weighing appropriate amounts of each compound, reconstituting in PBS, pH 7.4, sterile filtering the drug solution through a 0.22 □m PVDF syringe filter, and freezing aliquots for each day of dosing at −20° C.

Evaluation. Tumor size was monitored and body weight measured 3 times/week. Attention was given to gross animal morphology and behavior. Euthanasia was performed if the mice lost >20% of weight or when the tumors reached a size of 1500 mm3. Euthanasia was also performed at the researcher's discretion if mice lost a lot of weight in a short duration or when mice were approaching moribund conditions. Blood samples were collected by cardiac puncture from all viable animals from selected cohorts. Heart, lungs, liver, spleen, kidney, intestines, bone, brain and muscle were obtained immediately after euthanasia. The tissues were removed and stored in 10% neutral-buffered formalin.

As used herein, the term “partial response (PR)” refers to the observation of efficacy in a host animal, where the tested compound shows an improvement over control as measured by tumor volume during a predetermined observation period. For example, a 50% decrease in tumor volume from the initial measurement represents a partial response.

As used herein, the term “complete response (CR)” refers to the observation of efficacy in a host animal, where the tested compound shows an improvement over control by reducing the tumor volume to quantitation limits during a predetermined observation period. For example, a decrease in tumor volume where the measurements in two perpendicular directions are each about 2 mm or less represents a complete response.

As used herein, the term “cure (C)” refers to the observation of efficacy in a host animal, where the tested compound shows an improvement over control by reducing the tumor volume to quantitation limits, and where the tumor does not show significant regrowth during a predetermined observation period. For example, a decrease in tumor volume where the measurements in two perpendicular directions are each about 2 mm or less, and where the tumor does not regrow represents a cure.

### Example

As shown in FIGS. 5A and 5B, the efficacy of doxorubicin (DOXIL) was compared to EC1456 alone, and in combination with doxorubicin on vinca resistant KB-DR150 tumors in immunodeficient (XID) nu/nu mice deficient in NK cells. EC1456 showed improved efficacy over doxorubicin. The co-administration of EC1456 and doxorubicin showed an enhanced technical effect over both monotherapies.

Gross animal toxicity was not observed when EC1456 was administered alone. Mild toxicity was observed when doxorubicin was administered alone and in combination with EC1456. Without being bound by theory, it is believed herein that the mild toxicity observed with the combination therapy is attributable to doxorubicin.

### Example

As shown in FIGS. 6A and 6B, the efficacy of cisplatin was compared to EC1456 alone, and in combination with cisplatin on M109 (lung carcinoma) tumors in nu/nu mice. EC1456 showed improved efficacy over cisplatin. The co-administration of EC1456 and cisplatin showed an enhanced technical effect over both monotherapies.

Gross animal toxicity was not observed when EC1456 was administered alone. Mild toxicity was observed when cisplatin was administered alone and in combination with EC1456. Without being bound by theory, it is believed herein that the mild toxicity observed with the combination therapy is attributable to cisplatin.

### Example

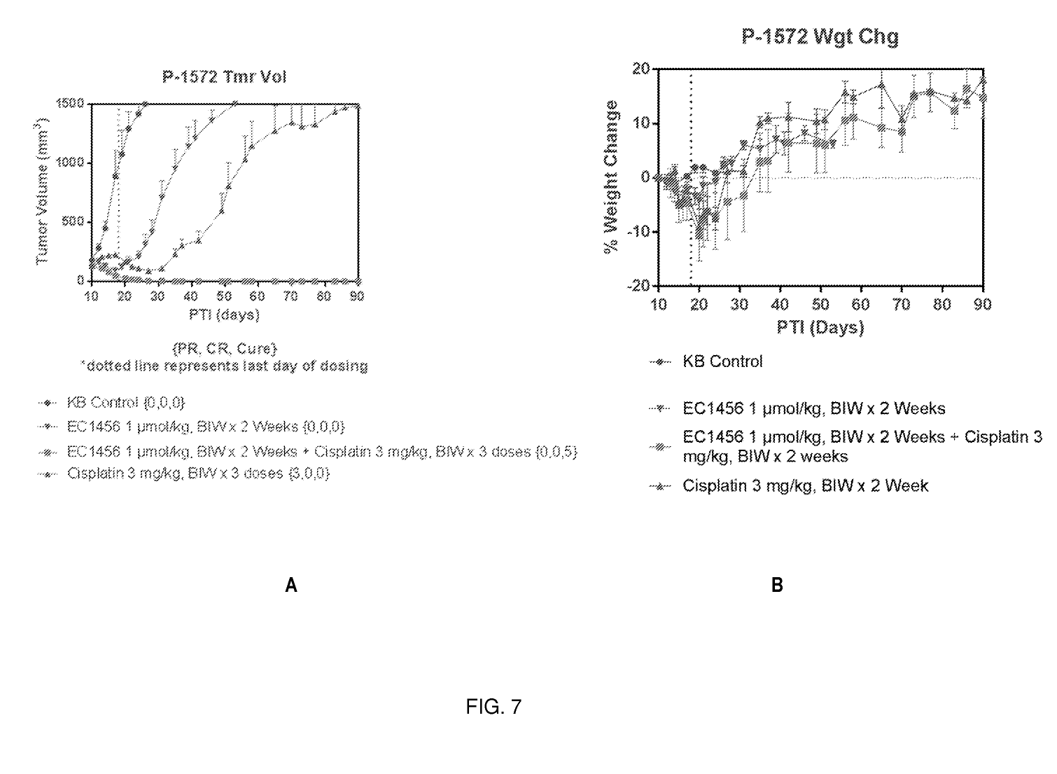

As shown in FIGS. 7A and 7B, the efficacy of cisplatin was compared to EC1456 alone, and in combination with cisplatin on KB tumors (oral epidermoid carcinoma) in nu/nu mice. EC1456 showed improved efficacy over cisplatin. The co-administration of EC1456 and cisplatin showed an enhanced technical effect over both monotherapies.

Gross animal toxicity was not observed when EC1456 was administered alone. Mild toxicity was observed when cisplatin was administered alone and in combination with EC1456. Without being bound by theory, it is believed herein that the mild toxicity observed with the combination therapy is attributable to cisplatin.

### Example

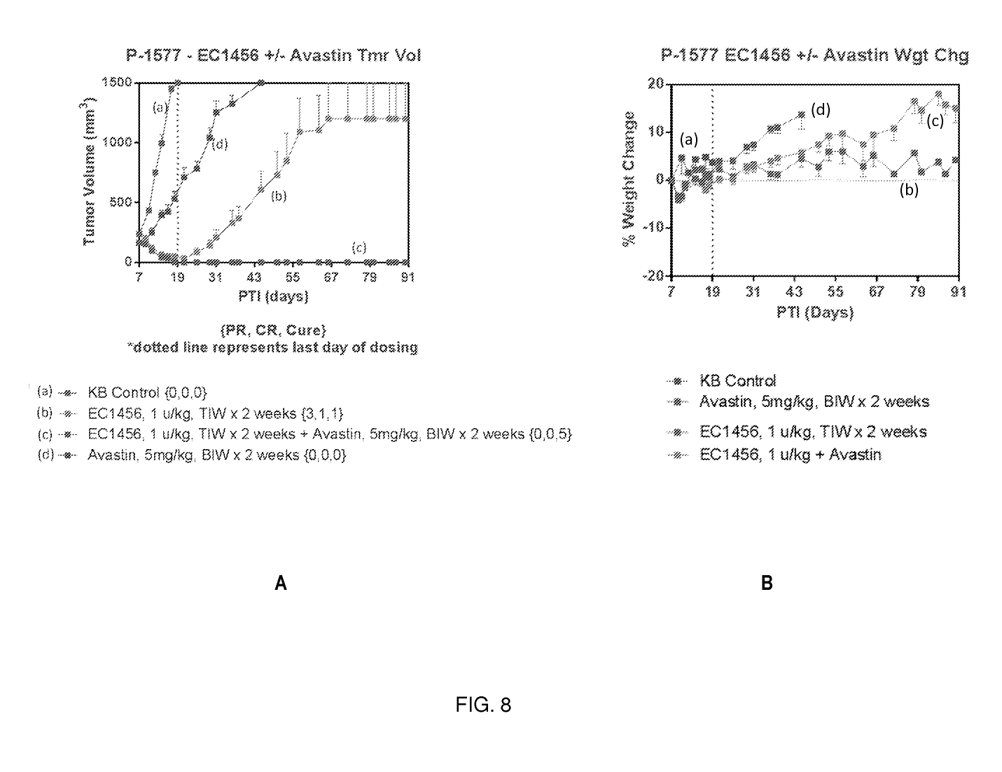

As shown in FIGS. 8A and 8B, the efficacy of bevacizumab was compared to EC1456 alone, and in combination with bevacizumab on KB tumors in nu/nu mice. EC1456 showed improved efficacy over bevacizumab. The co-administration of EC1456 and bevacizumab showed an enhanced technical effect over both monotherapies.

Gross animal toxicity was not observed with any of the mono or combination therapies.

### Example

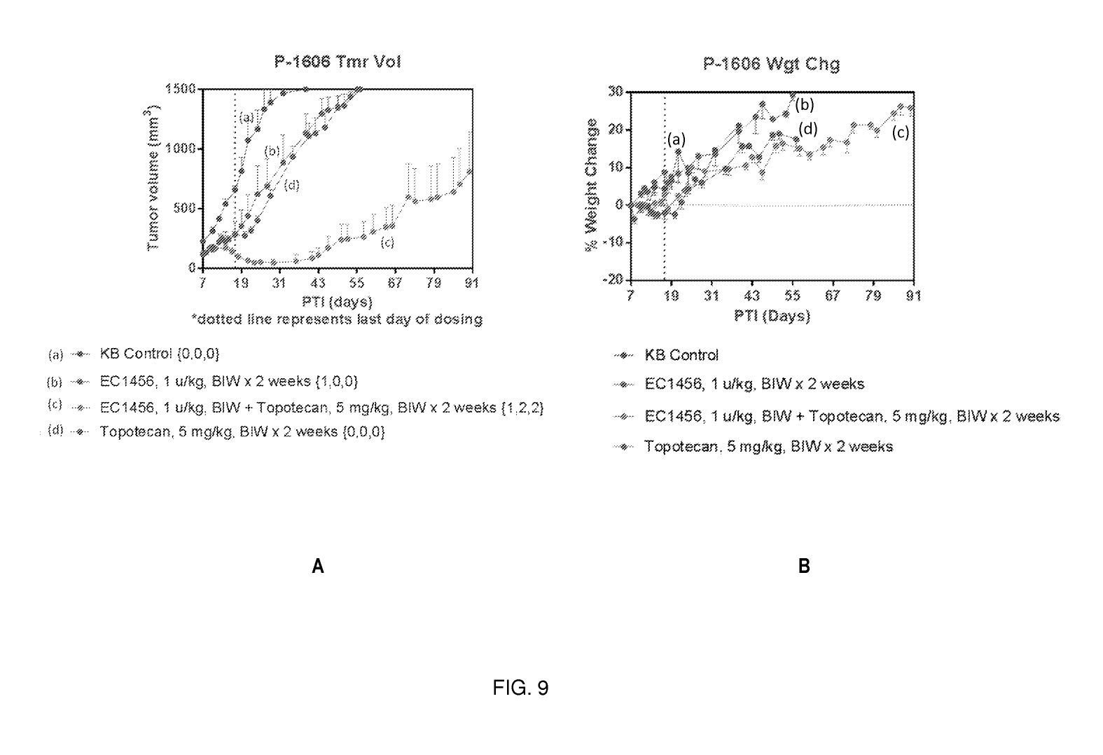

As shown in FIGS. 9A and 9B, the efficacy of topotecan was compared to EC1456 alone, and in combination with topotecan on KB tumors in nu/nu mice. EC1456 showed improved efficacy over topotecan. The co-administration of EC1456 and topotecan showed an enhanced technical effect over both monotherapies.

Gross animal toxicity was not observed with any of the mono or combination therapies.

### Example

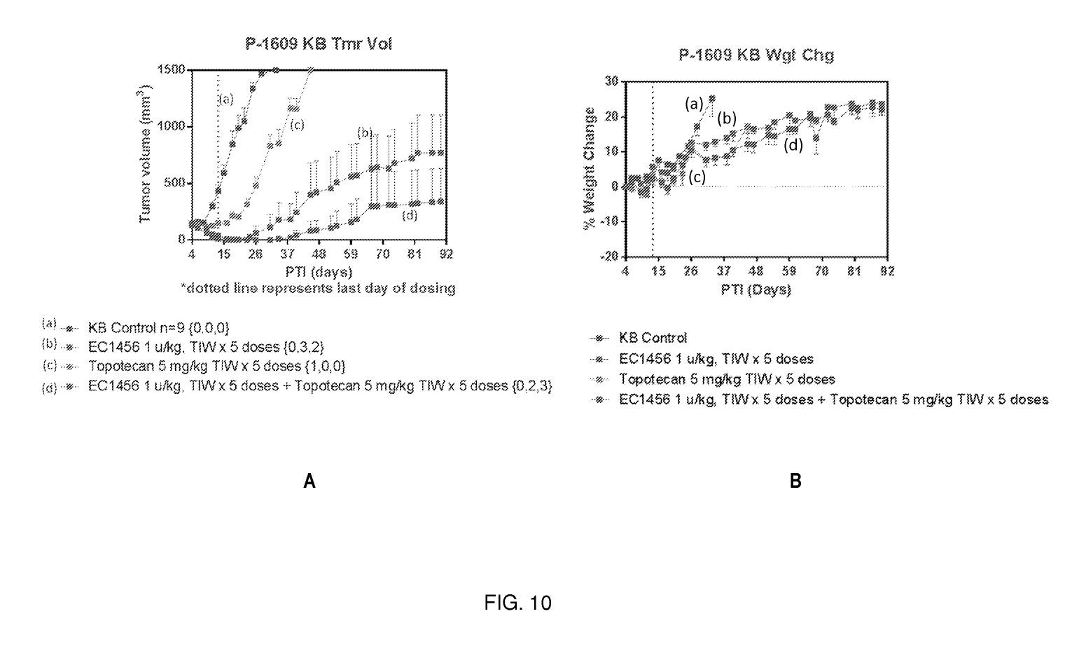

As shown in FIGS. 10A and 10B, the efficacy of topotecan was compared to EC1456 alone, and in combination with topotecan on KB tumors in nu/nu mice. EC1456 showed improved efficacy over topotecan. The co-administration of EC1456 and topotecan showed an enhanced technical effect over both monotherapies.

Gross animal toxicity was not observed with any of the mono or combination therapies.

### Example

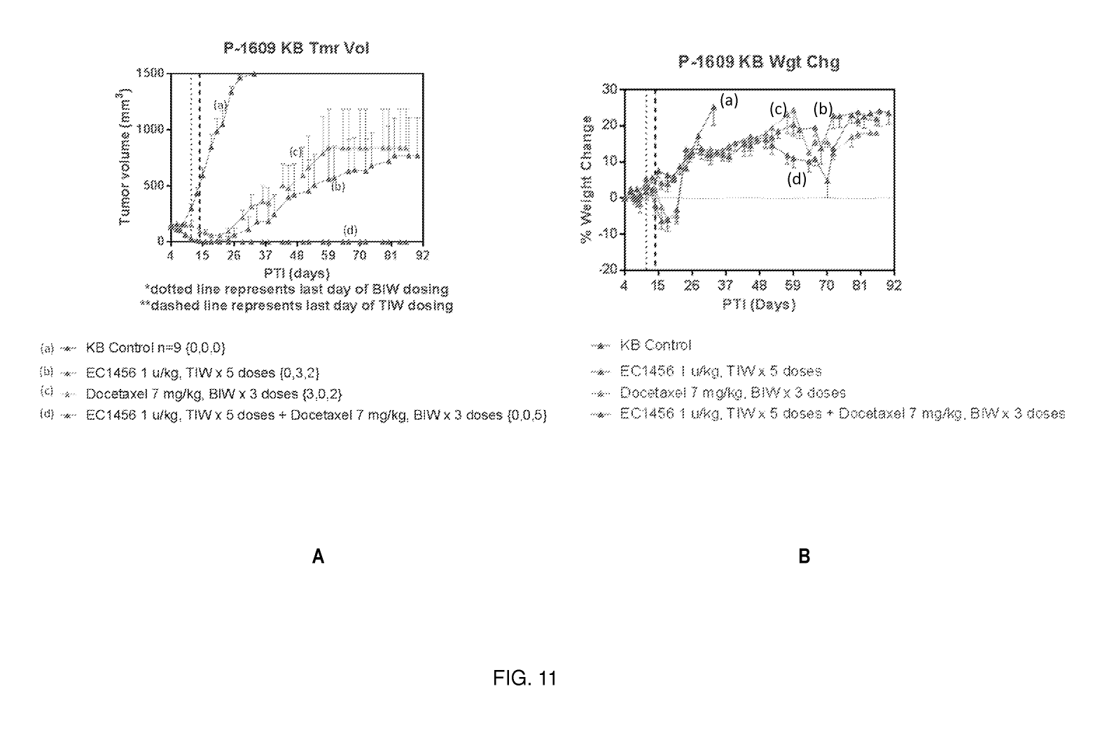

As shown in FIGS. 11A and 11B, the efficacy of docetaxel was compared to EC1456 alone, and in combination with docetaxel on KB tumors in nu/nu mice. EC1456 showed improved efficacy over docetaxel. The co-administration of EC1456 and docetaxel showed an enhanced technical effect over both monotherapies.

Gross animal toxicity was not observed when EC1456 was administered alone. Mild toxicity was observed when docetaxel was administered alone and in combination with EC1456. Without being bound by theory, it is believed herein that the mild toxicity observed with the combination therapy is attributable to docetaxel.

### Example

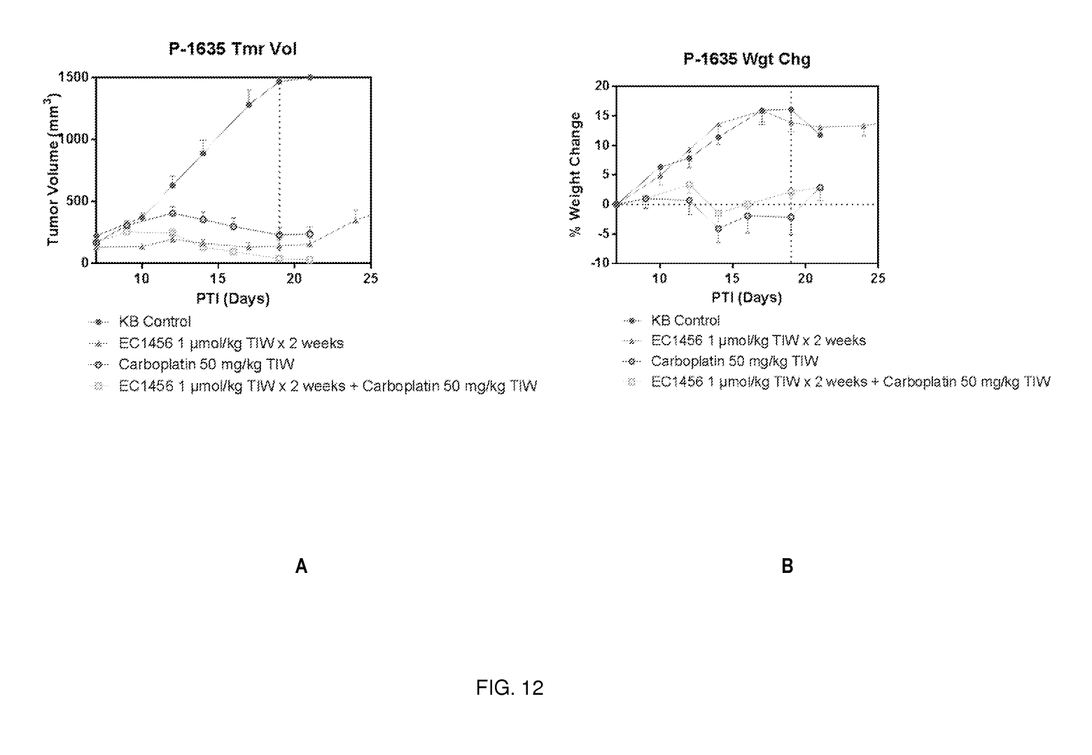

As shown in FIGS. 12A and 12B, the efficacy of carboplatin was compared to EC1456 alone, and in combination with carboplatin on KB tumors in nu/nu mice. EC1456 showed improved efficacy over carboplatin. The co-administration of EC1456 and carboplatin showed an enhanced technical effect over both monotherapies.

Gross animal toxicity was not observed when EC1456 was administered alone. Mild toxicity was observed when carboplatin was administered alone and in combination with EC1456. Without being bound by theory, it is believed herein that the mild toxicity observed with the combination therapy is attributable to carboplatin.

### Example

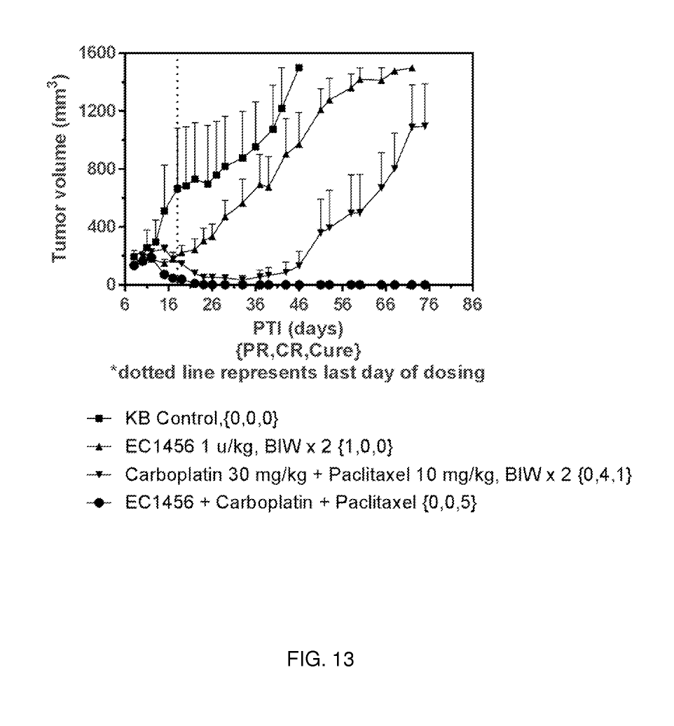

Antitumor effect of EC1456 in combination with Carboplatin/Paclitaxel on KB tumors. KB tumor cells (1×10⁶) were inoculated subcutaneously into nu/nu mice and therapy started on randomized mice. As shown in FIG. 13, each curve shows the average volume of 5 tumors. The efficacy of the combination of carboplatin with paclitaxel was compared to EC1456 alone, and in combination with the combination of carboplatin with paclitaxel on KB tumors in nu/nu mice. The co-administration of EC1456 and carboplatin and paclitaxel showed an enhanced technical effect over EC1456 alone and the combination of carboplatin with paclitaxel.

Gross animal toxicity was not observed when EC1456 was administered alone. Mild toxicity was observed when carboplatin was administered alone and in combination with EC1456. Without being bound by theory, it is believed herein that the mild toxicity observed with the combination therapy is attributable to carboplatin.

EC1456 at 1 μmol/kg on a twice a week for 2 weeks schedule showed minimum antitumor with 20% PR's. Carboplatin (30 mg/kg, BIW×2) when combined with paclitaxel (10 mg/kg, BIW×2) produced good anti-tumor activity with 80% CR's and 20% cures. However, EC1456 when added to the carboplatin/paclitaxel combination resulted in a far better anti-tumor activity with cures in 100% of the mice.

### Example

### Antitumor Activity in a PDX Tumor Models

As used herein, the term “stable disease (SD)” means no material progression of disease in a patient or animal over the course of therapy.

Female Balb/c nu/nu mice were fed ad libitum with folate-deficient chow (Harlan diet #TD01013) for the duration of the experiment. Primary human Endometrial model ST040, TNBC models 5T502 and ST738 and Ovarian model ST024 fragments (2-4 mm in diameter) were inoculated subcutaneously at the right flank of each mouse. Mice were randomized into 6 experimental groups of 5 or 3 mice each and test articles were injected through the lateral tail vein under sterile conditions in a volume of 200 μL of phosphate-buffered saline (PBS). These cancer cells were obtained from and studies were performed at South Texas Accelerated Research Therapeutics, 4383 Medical Drive, San Antonio, Tex. 78229

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm.

A. Endometrial PDX Tumor Model

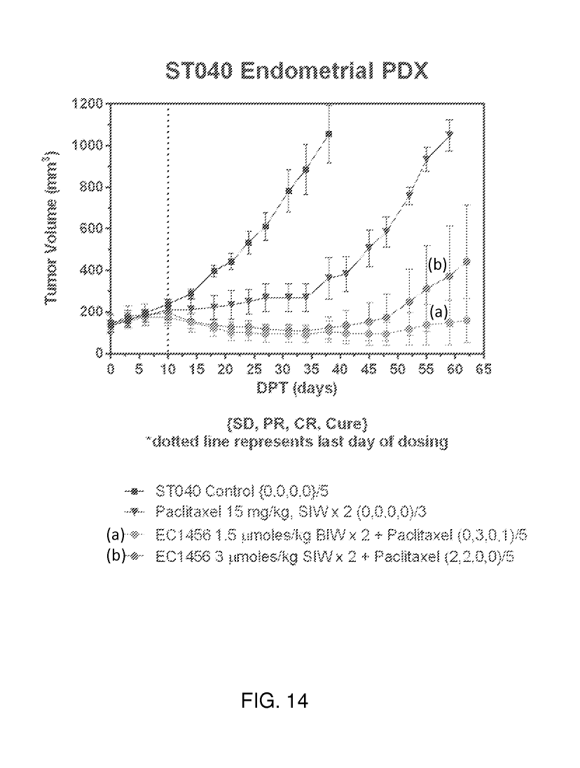

As shown in FIG. 14, treatment with 15 mg/kg of Paclitaxel (once a week for two weeks) produced minimal anti-tumor activity with zero animals exhibiting stable disease. When combined with Paclitaxel, EC1456 at 1.5 μmol/kg (two times a week for two weeks) produced good antitumor activity with 3 animals exhibiting PRs and 1 animal exhibiting cure while EC1456 at 3 μmol/kg (once a week for two weeks) produced 3 animals exhibiting stable disease and 2 animals exhibiting PR's.

B. TNBC PDX Tumor Models

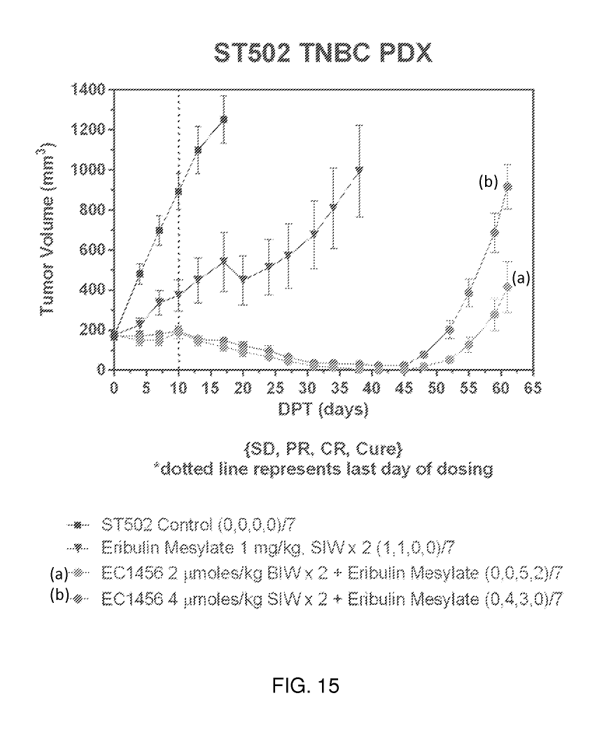

As shown in FIG. 15, treatment with 1 mg/kg of Eribulin mesylate (once a week for two weeks) produced minimal anti-tumor activity with 1 animal exhibiting stable disease/1 animal exhibiting PR. When combined with Eribulin mesylate, EC1456 produced synergistic antitumor activity at 2 μmol/kg (two times a week for two weeks) resulting in 5 animals exhibiting CRs and 2 animals exhibiting cures and at 4 μmol/kg (once a week for two weeks) generating 4 animals exhibiting PR's and 3 animals exhibiting CR's.

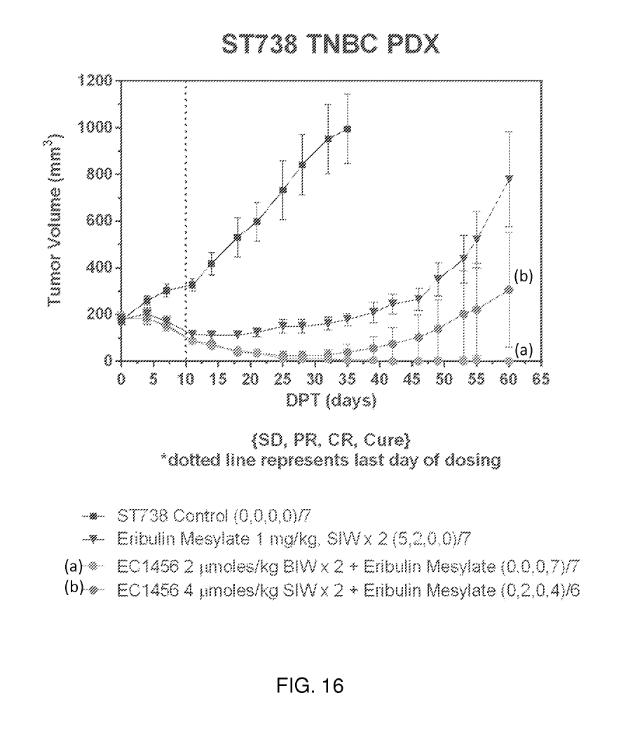

As shown in FIG. 16, treatment with 1 mg/kg of Eribulin mesylate (once a week for two weeks) produced some anti-tumor activity with 5 animals exhibiting stable disease/2 PR's. When combined with Eribulin mesylate, EC1456 produced curative antitumor activity with 2 μmol/kg (two times a week for two weeks) generating 7 animals exhibiting cures and 4 μmol/kg (once a week for two weeks) resulting in 2 animals exhibiting PR's and 4 animals exhibiting cures.

C. Ovarian PDX Tumor Model

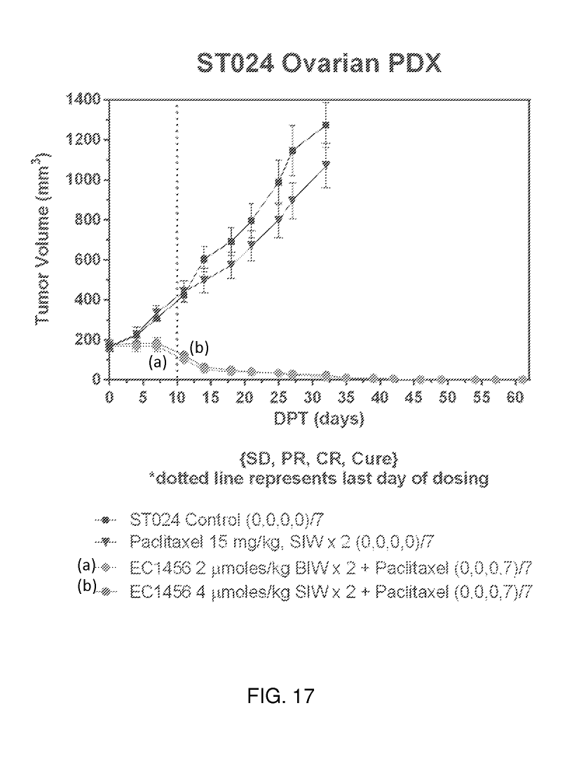

As shown in FIG. 17, treatment with 15 mg/kg of Paclitaxel (once a week for two weeks) produced no anti-tumor activity. EC1456 at 2 μmol/kg (two times a week for two weeks) and 4 μmol/kg (once a week for two weeks) produced curative (100% animals exhibiting cures) anti-tumor activity when combined with Paclitaxel.

### Example

### Antitumor Activity in Huprime® NSCLC PDX Tumor Models

Female Balb/c nu/nu mice were fed ad libitum with folate-deficient chow (Harlan diet #TD01013) for the duration of the experiment. Primary human NSCLC models LU1147 or LU2505 fragments (2-4 mm in diameter) were inoculated subcutaneously at the right flank of each mouse. Mice were randomized into experimental groups of 7 mice and test articles were injected through the lateral tail vein under sterile conditions in a volume of 200 μL of phosphate-buffered saline (PBS). These studies were performed at Crown Bioscience (Beijing) Inc., Ground Floor, Light Muller Building, Changping Sector of Zhongguancun Scientific Park, No. 21 Huoju Road, Changping District, Beijing, P.R. China.

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm.

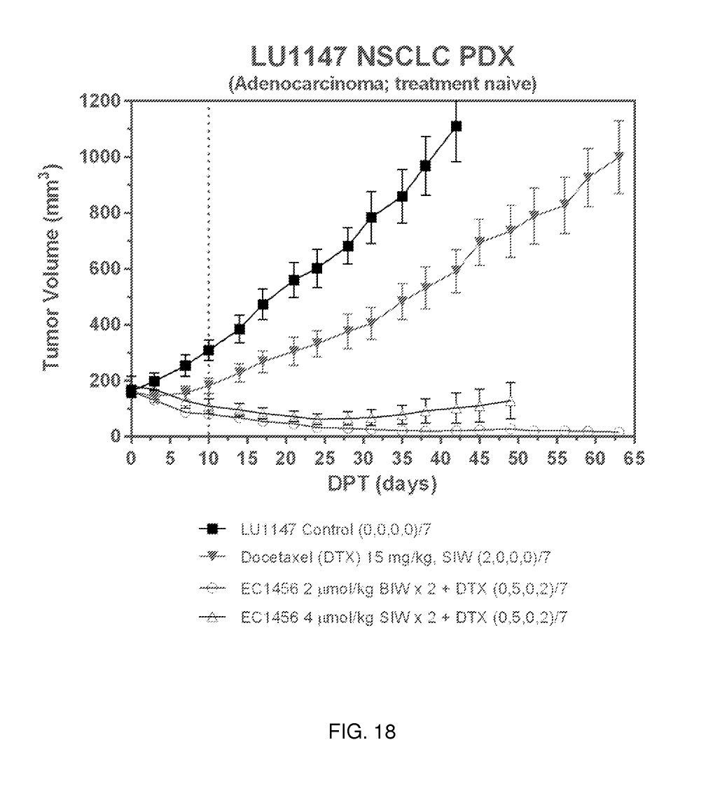

As shown in FIG. 18, treatment with 15 mg/kg of Docetaxel (one dose) produced minimal anti-tumor activity with 2 animals exhibiting stable disease. When combined with Docetaxel (one dose of 15 mg/kg), EC1456 at 2 μmol/kg (two times a week for two weeks) and 4 μmol/kg (once a week for two weeks) produced good antitumor activity in animals exhibiting stable disease with 5 PRs and 2 cures in both groups.

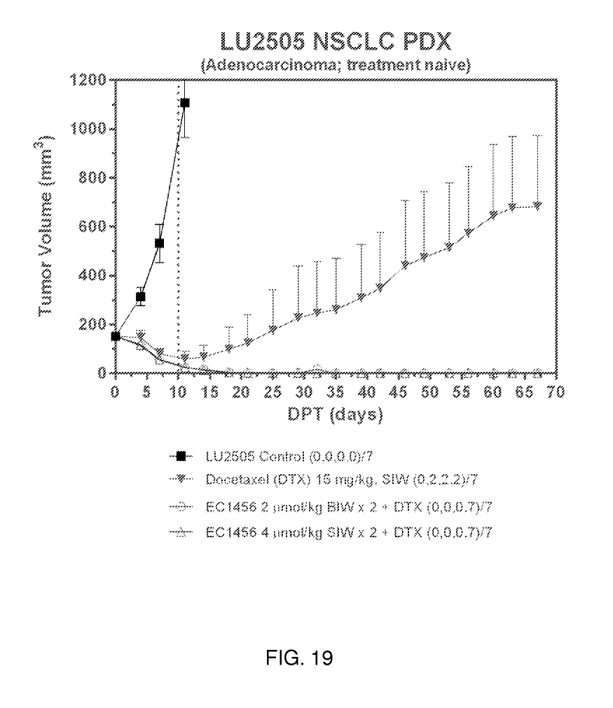

As shown in FIG. 19, treatment with 15 mg/kg of Docetaxel (one dose) produced some anti-tumor activity in animals exhibiting stable disease with 2 PR's, 2 CR's and 2 cures. However, EC1456 at 2 μmol/kg (two times a week for two weeks) and 4 μmol/kg (once a week for two weeks) in combination with Docetaxel produced 100% cures in both groups of animals exhibiting stable disease.

1. A method for treating cancer in a host animal, the method comprising the step of administering to the host animal a therapeutically effective amount of a compound of the formula 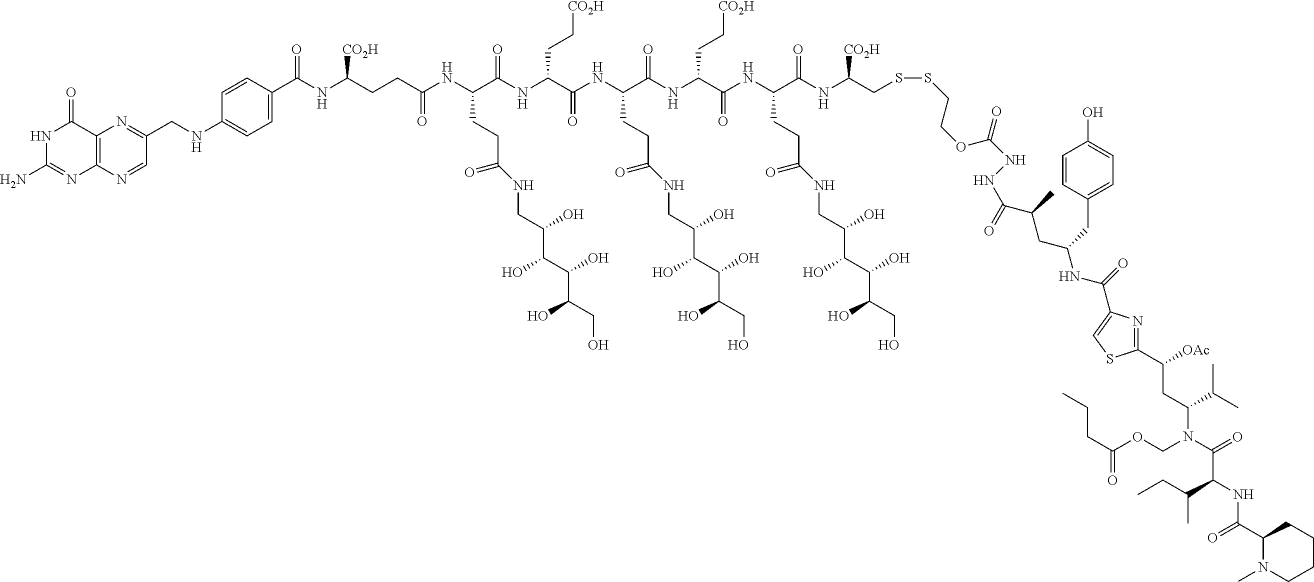 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]
or a pharmaceutically acceptable salt thereof,
in combination with a therapeutically effective amount of at least one additional anti-cancer agent.

2. The method of claim 1, wherein the cancer is selected from the group consisting of a carcinoma, a sarcoma, a lymphoma, Hodgekin's disease, a melanoma, a mesothelioma, Burkitt's lymphoma, a nasopharyngeal carcinoma, a leukemia, and a myeloma.

3. The method of claim 1, wherein the cancer is selected from the group consisting of oral cancer, thyroid cancer, endometrial cancer, endocrine cancer, skin cancer, gastric cancer, esophageal cancer, laryngeal cancer, pancreatic cancer, colon cancer, bladder cancer, bone cancer, ovarian cancer, cervical cancer, uterine cancer, breast cancer, testicular cancer, prostate cancer, rectal cancer, kidney cancer, liver cancer, and lung cancer.

4. The method of claim 1, wherein the cancer is ovarian cancer.

5. The method of claim 1, wherein the cancer is non-small cell lung cancer.

6. The method of claim 1, wherein the cancer is endometrial cancer.

7. The method of claim 1, wherein the cancer is triple negative breast cancer.

8. The method of claim 1, wherein the cancer is breast cancer.

9. The method of claim 1, wherein the cancer is lung cancer.

10. The method of claim 1, wherein the additional anti-cancer agent is selected from the group consisting of doxorubicin (DOXIL), cisplatin, bevacizumab (Avastin), topotecan, eribulin mesylate, docetaxel, paclitaxel, and carboplatin, or a pharmaceutically acceptable salt thereof.

11. The method of claim 10, wherein the additional anti-cancer agent is selected from the group consisting of eribulin mesylate, docetaxel and paclitaxel, or a pharmaceutically acceptable salt thereof.

12. The method of claim 10, wherein the additional anti-cancer agent is doxorubicin (DOXIL), or a pharmaceutically acceptable salt thereof.

13. The method of claim 10, wherein the additional anti-cancer agent is cisplatin, or pharmaceutically acceptable salt thereof.

14. The method of claim 10, wherein the additional anti-cancer agent is bevacizumab (Avastin), or a pharmaceutically acceptable salt thereof.

14. The method of claim 10, wherein the additional anti-cancer agent is eribulin mesylate.

16. The method of claim 10, wherein the additional anti-cancer agent is docetaxel, or a pharmaceutically acceptable salt thereof.

17. The method of claim 10, wherein the additional anti-cancer agent is paclitaxel, or a pharmaceutically acceptable salt thereof.

18. The method of claim 10, wherein the additional anti-cancer agent is carboplatin, or a pharmaceutically acceptable salt thereof.

19.-38. (canceled)
